SEQUENCE LISTING (22 sequences)

SEQ ID NO: 1 - DNA, 897 bp, Artificial Sequence
  Sequencing cassette
  misc_feature (1)...(76): Adaptor sequence
  misc_feature (77)...(90): 14 nucleotide bar code
  misc_feature (77)...(90): n = A, T, C or G
  misc_feature (91)...(105): XcmI restriction site
  misc_feature (106)...(882): GFP insert
caagcagaagacggcatacgagatctagtacgcggtctgccttgccagcccgctcagagatgtgtataagagacagnnnnnnnnnnnnnnccaatactcgagtggttgacagctagctcagtcctaggtataatgctagccaatttcacaaggaggcagctaatggtgagcaagggcgaggagctgttcaccggggtggtgcccatcctggtcgagctggacggcgacgtgaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaaccataccatggttgg

SEQ ID NO: 2 - DNA, 21 bp, Artificial Sequence
  Primer
caagcagaagacggcatacga

SEQ ID NO: 3 - DNA, 92 bp, Artificial Sequence
  Targeting primer
aatgatacggcgaccaccgagatctacactagatcgcgcctccctcgcgccatcagagatgtgtataagagacaggctcatggtgggggcag

SEQ ID NO: 4 - DNA, 20 bp, Artificial Sequence
  Primer
aatgatacggcgaccaccga

SEQ ID NO: 5 - DNA, 7 bp, Artificial Sequence
  7 nucleotide barcode
  misc_feature (1)...(7): n = A,T,C or G
nnnnnnn

SEQ ID NO: 6 - DNA, 3568 bp, Artificial Sequence
  Vector Sequence with GFP insert
  misc_feature (402)...(477): Adaptor sequence
  misc_feature (478)...(491): 14 nucleotide bar code
  misc_feature (478)...(491): n = A,T,C or G
  misc_feature (492)...(506): XcmI restriction site
  misc_feature (507)...(1283): GFP insert
  misc_feature (1284)...(1298): XcmI restriction site
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattccaagcagaagacggcatacgagatctagtacgcggtctgccttgccagcccgctcagagatgtgtataagagacagnnnnnnnnnnnnnnccaatactcgagtggttgacagctagctcagtcctaggtataatgctagccaatttcacaaggaggcagctaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccataccatggttggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 7 - DNA, 33 bp, Artificial Sequence
  Sense strand index sequence
  misc_feature (16)...(18): non-complementary sequence
gcatagcgcatgcagccccactagcgactcgat

SEQ ID NO: 8 - DNA, 32 bp, Artificial Sequence
  Antisense strand index sequence
  misc_feature (16)...(18): non-complementary sequence
tcgagtcgctagtgcttttgcatgcgctatgc

SEQ ID NO: 9 - DNA, 76 bp, Artificial Sequence
  Nextera Adaptor Sequence
caagcagaagacggcatacgagatctagtacgcggtctgccttgccagcccgctcagagatgtgtataagagacag

SEQ ID NO: 10 - DNA, 75 bp, Artificial Sequence
  Nextera Adaptor Sequence
aatgatacggcgaccaccgagatctacactagatcgcgcctccctcgcgccatcagagatgtgtataagagacag

SEQ ID NO: 11 - DNA, 24 bp, Artificial Sequence
  Nextera Adaptor Sequence
caagcagaagacggcatacgagat

SEQ ID NO: 12 - DNA, 29 bp, Artificial Sequence
  Nextera Adaptor Sequence
aatgatacggcgaccaccgagatctacac

SEQ ID NO: 13 - DNA, 30 bp, Artificial Sequence
  Ion Xpress Adaptor Sequence 1
ccatctcatccctgcgtgtctccgactcag

SEQ ID NO: 14 - DNA, 23 bp, Artificial Sequence
  Ion Xpress Adaptor Sequence 2
cctctctatgggcagtcggtgat

SEQ ID NO: 15 - DNA, 25 bp, Artificial Sequence
  Roche 454 Adaptor Sequence 1
cgtatcgcctccctcgcgccatcag

SEQ ID NO: 16 - DNA, 25 bp, Artificial Sequence
  Roche 454 Adaptor Sequence 2
ctatgcgccttgccagcccgctcag

SEQ ID NO: 17 - DNA, 14 bp, Artificial Sequence
  14 nucleotide barcode
  misc_feature (1)...(14): n = A,T,C or G
nnnnnnnnnnnnnn

SEQ ID NO: 18 - DNA, 20 bp, Artificial Sequence
  p53 exon 5-6 Forward primer
cacttgtgccctgactttca

SEQ ID NO: 19 - DNA, 20 bp, Artificial Sequence
  p53 exon 5-6 Reverse primer
cttaacccctcctcccagag

SEQ ID NO: 20 - DNA, 50 bp, Artificial Sequence
  Nested p53 exon 5 primer
  misc_feature (1)...(33): Adaptor read primer
  misc_feature (34)...(50): p53 exon 5 specific sequence
tcgtcggcagcgtcagatgtgtataagagacaggctcatggtgggggcag

SEQ ID NO: 21 - DNA, 51 bp, Artificial Sequence
  Nextera Index primer S505
  misc_feature (1)...(29): flow cell adaptor sequence
  misc_feature (30)...(37): index sequence
  misc_feature (38)...(51): Adaptor read primer
aatgatacggcgaccaccgagatctacacgtaaggagtcgtcggcagcgtc

SEQ ID NO: 22 - DNA, 51 bp, Artificial Sequence
  Nextera Index primer S506
  misc_feature (1)...(29): flow cell adaptor sequence
  misc_feature (30)...(37): index sequence
  misc_feature (38)...(51): Adaptor read primer
aatgatacggcgaccaccgagatctacacactgcatatcgtcggcagcgtc

(19) United States
(12) Patent Application Publication
(10) Pub. No.: US 2018/0119214 A1
(43) Pub. Date: May 3, 2018

(54) COMPOSITIONS AND METHODS FOR TARGET NUCLEIC ACID MOLECULE ENRICHMENT

(71) Applicant: FRED HUTCHINSON CANCER RESEARCH CENTER, Seattle, WA (US)

(72) Inventors: Jason H. BIELAS, Seattle, WA (US); Mark T. GREGORY, Seattle, WA (US)

(21) Appl. No.: 15/560,893

(22) PCT Filed: Mar. 31, 2016
(86) PCT No.: PCT/US2016/025366

§ 371 Date: Sep. 22, 2017

Related U.S. Application Data
(60) Provisional application No. 62/140,930, filed on Mar. 31, 2015.

(51) Int. Cl. C12Q 1/6858; C12N 15/10; C12Q 1/6855; C12Q 1/6886

(52) U.S. Cl. CPC C12Q 1/6858; C12N 15/1065; C12Q 2600/156; C12Q 1/6886; C12Q 1/6855

(57) ABSTRACT

The present disclosure relates to methods of detecting mutations in a target nucleic acid molecule by amplifying a target sequence to contain adaptor sequences and allowed for the product to be directly sequenced using high throughput DNA sequencing technology.

### STATEMENT OF GOVERNMENT INTEREST

This invention was made with government support under R01 ES019319 awarded by the National Institutes of Health. The government has certain rights in the invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360056_432WO_SEQUENCE_LISTING.txt. The text file is 11.7 KB, was created on Mar. 31, 2016, and is being submitted electronically via EFS-Web.

### BACKGROUND

The Food and Drug Administration (FDA) has recently approved the use of four diagnostic devices comprising two cystic fibrosis assays, kit reagents, and the Illumina MiSeqDx platform for high throughput gene sequencing, commonly referred to as next-generation sequencing (NGS) (Sheridan, Nat. Biotechnol. 32:111-112, 2014). The decision by the FDA has paved the way for future clinical diagnostic and prognostic use of NGS in a multitude of disease settings, including cancer. With ongoing advances in personalized medicine, there is a need to understand what spontaneous or rare mutations may be contributing to a disease.

Detection of spontaneous mutations (e.g., substitutions, insertions, deletions, duplications), or even induced mutations, that occur randomly throughout a genome can be challenging because these mutational events are rare and may exist in one or only a few copies of DNA. The most direct way to detect mutations is by sequencing, but the available sequencing methods are not sensitive enough to detect rare mutations. For example, mutations that arise de novo in mitochondrial DNA (mtDNA) will generally only be present in a single copy of mtDNA, which means these mutations are not easily found since a mutation must be present in as much as 10-25% of a population of molecules to be detected by sequencing (Jones et al., Proc. Nat'l. Acad. Sci. U.S.A. 105:4283-88, 2008). As another example, the spontaneous somatic mutation frequency in genomic DNA has been estimated to be as low as 1×10⁻⁸ and 2.1×10⁻⁶ in human normal and cancerous tissues, respectively (Bielas et al., Proc. Nat'l Acad. Sci. U.S.A. 103:18238-42, 2008).

One improvement in sequencing has been to take individual DNA molecules and amplify the number of each molecule by, for example, polymerase chain reaction (PCR) and digital PCR. Indeed, massively parallel sequencing represents a particularly powerful form of digital PCR because multiple millions of template DNA molecules can be analyzed one by one. However, the amplification of single DNA molecules prior to or during sequencing by PCR and/or bridge amplification suffers from the inherent error rate of polymerases employed for amplification, and spurious mutations generated during amplification may be misidentified as spontaneous mutations from the original (endogenous unamplified) nucleic acid. Similarly, DNA templates damaged during preparation (ex vivo) may be amplified and incorrectly scored as mutations by massively parallel sequencing techniques. Again, using mtDNA as an example, experimentally determined mutation frequencies are strongly dependent on the accuracy of the particular assay being used (Kraytsberg et al., Methods 46:269-73, 2008)—these discrepancies suggest that the spontaneous mutation frequency of mtDNA is either below, or very close to, the detection limit of these technologies. Massively parallel sequencing cannot generally be used to detect rare variants because of the high error rate associated with the sequencing process—one process using bridge amplification and sequencing by synthesis has shown an error rate that varies from about 0.06% to 1%, which depends on various factors including read length, base-calling algorithms, and the type of variants detected (see Kinde et al., Proc. Nat'l. Acad. Sci. U.S.A. 108:9530-5, 2011).

Accordingly, there is a need for methods that allow for rapid and error corrected detection of mutations in samples with high levels of background sequence. The present disclosure meets such needs, and further provides other related advantages.

### BRIEF DESCRIPTION OF THE DRAWINGS

FIGS. 1A and 1B are graphical depictions of an exemplary method of generating a genomic DNA library and polymerase chain reaction (PCR) enrichment. (A) A vector backbone that includes a first adaptor sequence, a barcode (e.g., a 14-mer barcode as set forth in SEQ ID NO:17), and two XcmI restriction site flanking an insert is cleaved using an XcmI restriction enzyme. A fragmented segment of genomic DNA is cloned into the vector via A-overhangs complementary to T-overhangs in the vector created by XcmI, using DNA ligase. (B) A genomic DNA plasmid library is used as a template for a PCR reaction using a library primer that includes an adaptor sequence and a target-specific primer that includes a second adaptor sequence. The amplification products can be sequenced using a next generation sequencing technology (e.g., Illumina sequencing).

FIGS. 2A and 2B are graphical depictions of other exemplary methods of generating a genomic DNA library and PCR enrichment. (A) A vector backbone that includes a first adaptor sequence, a barcode (e.g., a 14-mer barcode as set forth in SEQ ID NO:17 and two restriction sites flanking an insert (e.g., XcmI at the 3′ end of the insert and another restriction site at the 5′ end of the insert) is cleaved using XcmI and another restriction enzyme, resulting in linearized vectors with one 3′-end T-overhang and a non-complementary overhang at the 5′-end. Fragmented segments of genomic DNA that have been A-tailed by Taq polymerase and a strand index sequence (SIS) that has one 3′-end T-overhang and a 5′-end overhang that is complementary to the 5′-end overhang of the vector are cloned into the vector using DNA ligase. (B) (i) A genomic DNA plasmid library is used as a template for a PCR reaction using a library primer that includes the first adaptor sequence and a non-targeting primer that hybridizes to the vector backbone and includes a second adaptor sequence, optionally biotinylated, to amplify all genomic DNA fragments represented in the library. (ii) Alternatively, the genomic DNA plasmid library prepared as shown in FIG. 1A or FIG. 2A is used as a template for a PCR reaction using a library primer that includes the first adaptor sequence and a target-specific primer that does not include a second adaptor Sequence. The amplification products are used in a subsequent round of PCR using a nested target-specific primer that hybridizes to a target region that is located 5′ to the target-specific primer. The nested target-specific primer includes a second adaptor sequence that is optionally biotinylated. (iii) In another example, the genomic DNA plasmid library is used as a template for a PCR reaction using a library primer that includes the first adaptor sequence and a target-specific primer that includes a second adaptor sequence that is optionally biotinylated. The amplification products from the various embodiments can be sequenced using a next generation sequencing technology, and the strand specificity of each sequence is determined based on the strand index sequence.

FIG. 3 shows that a rare target (p53) can be amplified from a genomic library sample using PCR. PCR reactions were performed using a library primer and a p53 exon 4 specific targeting primer on three genomic library samples as follows: 12.5 ng HeLa library alone, 12.5 ng HeLa library spiked with 0.125 pg p53 library, and 0.125 pg p53 library alone. The PCR products from each reaction were size separated on an agarose gel. The lanes labeled with a (−) show untreated PCR amplification products, whereas the lanes labeled with a (+) show PCR products that have been treated with a NcoI restriction endonuclease.

FIG. 4 is a bar graph depicting the enrichment of a HCT116 genomic library and a HCT116 library spiked with a p53 library for exon 5 and exon 6 regions. The libraries were prepared using the methods described herein and amplified using a library primer that includes a first adaptor sequence and a biotinylated target-specific primer. Following affinity purification with the biotin tag, the amplification products were used in a subsequent round of PCR using the library primer and a nested target-specific primer that includes an adaptor read primer sequence. These amplicons were templates for another round of PCR with the library primer and “index” primers comprising a flow cell adaptor sequence, an index sequence, and a read primer sequence, with a different index primer used for each sample. The index primer appended unique read indices to the target nucleic acid molecules to each sample and allowed for multiplex sequencing. Enrichment was quantified by droplet digital PCR and next generation sequencing.

FIG. 5 is a scatter plot depicting the error-corrected mutation frequency for the the p53 exon 5 region in HCT116 genomic library sample and HCT116 library spiked with a p53 library for exon 5 and exon 6 regions. Double-stranded bar codes present within the sequencing reads were used to create a consensus sequence from each read family. A mutation is considered real and not an artifact (“called”) if it is observed in two or more read families.

FIG. 6 is a scatter plot depicting the error rate in sequencing data for p53 exon 5 region in HCT116 genomic library sample and HCT116+p53 spike-in sample, without the benefit of error correction using the double-stranded molecular bar codes.

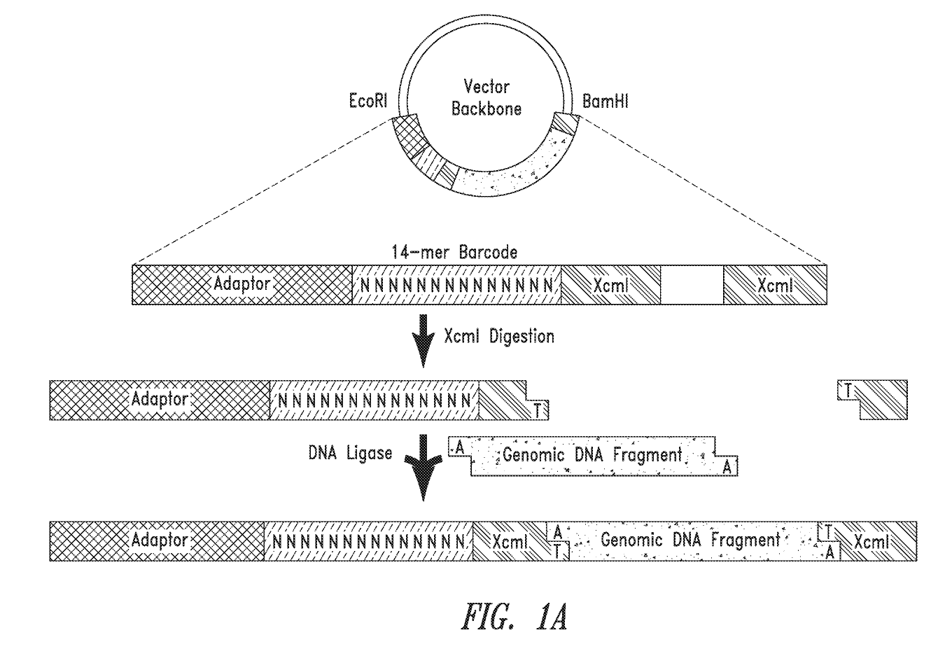

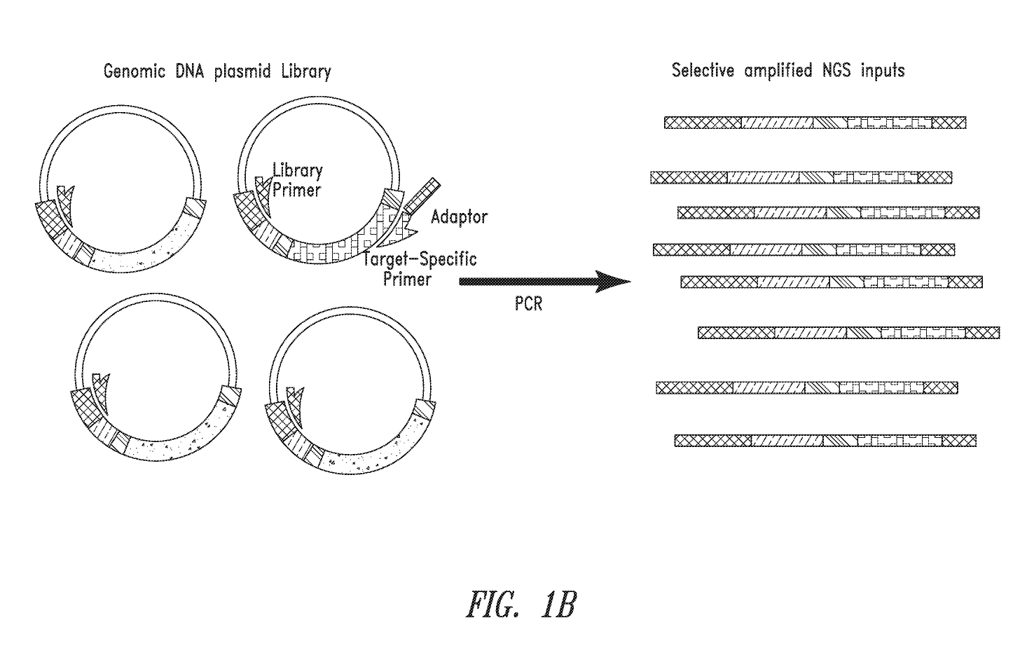

### DETAILED DESCRIPTION

The present disclosure provides a high fidelity method of detecting one or more mutations in a target nucleic acid molecule. The compositions and methods provided herein allow for quick and accurate detection of rare mutations in polynucleotides of interest by reducing sample handling time, and reducing the potential sequencing error rate associated with existing sequencing methods.

Accordingly, one aspect of the present disclosure provides a method for detecting mutations in a target nucleic acid molecule by using, for example, the polymerase chain reaction (PCR) to amplify a template target nucleic acid molecule contained in a vector with a barcode, a first adaptor, and a first priming site. A target nucleic acid molecule can be any sequence, region or fragment of interest (e.g., encodes a biological molecule of interest or fragment thereof) that can be contained in, for example, a genomic library. Each of the plurality of nucleic acid molecules of a library can be flanked on one end by a first priming site, a first adaptor region and a barcode. A target nucleic acid molecule from such a genomic library or fragment thereof can be amplified with a plurality of primers that includes (1) a library primer that has at least 80% sequence identity with a first priming site that is adjacent to or near a target nucleic acid molecule (e.g., a library primer's first priming site may on a vector), and (2) a targeting primer that has at least 80% sequence identity with a target nucleic acid molecule or a portion of the target nucleic acid molecule. The targeting primer optionally includes a second adaptor sequence. Thus, a library primer can be used together with a targeting primer that contains the second adaptor sequence to sequence an amplified target nucleic acid molecule. Alternatively, the library primer and targeting primer may be used to amplify the target nucleic acid molecule, and the resulting amplicons used as template for a subsequent round of PCR using the library primer and a nested targeting primer that contains the second adaptor sequence. The resulting amplicons from the nested PCR reaction may then be sequenced. For enrichment of all the nucleic acid molecules present in a library, a target nucleic acid molecule can be amplified with a plurality of primers that includes (1) a library primer that has at least about 90% sequence identity with a first priming site that is adjacent to or near a target nucleic acid molecule (e.g., a library primer's first priming site may on a vector), and (2) a non-targeting primer that has at least about 90% sequence identity with a second priming site that is adjacent to or near the target nucleic acid molecule on the opposite end from the library primer, first adaptor region and the bar code. The non-targeting primer optionally includes a second adaptor sequence. Thus, the library primer and the non-targeting primer that contains the second adaptor sequence can be used to enrich or sequence all the fragments in the library, or both. The sequence can be analyzed for the presence of mutation(s). The barcodes can be used to computationally deconvolute the sequencing data and map all sequence reads to single molecules (i.e., distinguish PCR and sequencing errors from real mutations). A strand index sequence can be incorporated in the library and used in combination with the barcodes to map all sequence reads to a specific strand of a single nucleic acid molecule. Base calling and sequence alignment can be performed using, for example, the Eland pipeline (Illumina, San Diego, Calif.).

The methods and compositions disclosed herein can be used, for example, in the field of personalized medicine because the methods allow for the rapid identification of specific mutations that may be contributing to disease progression in a particular patient. Accordingly, a precision therapy, treatment or therapeutic regimen can be developed based on a patient's specific genotype.

The compositions and methods of this disclosure allow a person of skill in the art to rapidly and more accurately sequence target nucleic acid molecules of interest while distinguishing true mutations (i.e., naturally arising in vivo mutations) from artifact “mutations” (i.e., ex vivo mutations that may arise for various reasons, such as a downstream amplification error, a sequencing error, or physical or chemical damage). If a mutation pre-existed in an original double-stranded nucleic acid molecule before isolation, amplification or sequencing, then, for example, a transition mutation of adenine (A) to guanine (G) identified on one strand will be complemented with a thymine (T) to cysteine (C) transition identified on the other strand. In contrast, artifact “mutations” that arise later on an individual (separate) DNA strand due to polymerase errors during isolation, amplification or sequencing are extremely unlikely to have a matched base change in the complementary strand. The approach of this disclosure provides compositions and methods for interrogating one or more regions within a target nucleic acid molecule, or interrogating one or more target nucleic acid molecules in a multiplex reaction and distinguishing systematic errors (e.g., polymerase read fidelity errors) and biological errors (e.g., chemical or other damage) from actual known or newly identified mutations or single nucleotide polymorphisms (SNPs).

By way of background, any spontaneous or induced mutation will be present in both strands of a native genomic, double-stranded DNA molecule. Hence, such a mutant DNA template amplified using error-free PCR would result in a PCR product in which 100% of the molecules produced by PCR include the mutation. In contrast to an original, spontaneous mutation, a change due to polymerase error will only appear in one strand of the initial template DNA molecule (while the other strand will not have the artifact mutation). If all DNA strands in a PCR reaction are copied equally efficiently, then any polymerase error that emerged at the first PCR cycle likely will be found in at least 25% of the total PCR product. But DNA molecules or strands are not copied equally efficiently, so DNA sequences amplified from the strand that incorporated an erroneous nucleotide base during the initial amplification might constitute more or less than 25% of the population of amplified DNA sequences depending on the efficiency of amplification. Similarly, any polymerase error that occurs in later PCR cycles will generally represent an even smaller proportion of PCR products (e.g., 12.5% for the second cycle, 6.25% for the third, etc.). PCR-induced mutations may be due to polymerase errors or due to the polymerase bypassing damaged nucleotides, thereby resulting in an error (see, e.g., Bielas and Loeb, Nat. Methods 2:285-90, 2005). For example, a common change to DNA is the deamination of cytosine, which is recognized by Taq polymerase as a uracil and results in a cytosine to thymine transition mutation (Zheng et al., Mutat. Res. 599:11-20, 2006)—that is, an alteration in the original DNA sequence may be detected when the damaged DNA is sequenced, but such a change may or may not be recognized as a sequencing reaction error or due to damage arising ex vivo (e.g., during or after nucleic acid isolation).

Due to potential artifacts and alterations of nucleic acid molecules arising from isolation, amplification and sequencing, the accurate identification of true somatic DNA mutations is difficult when sequencing amplified nucleic acid molecules. Consequently, evaluation of whether certain mutations are related to, or are a biomarker for, various disease states (e.g., cancer) or aging becomes confounded.

Next generation sequencing provides a means for sequencing multiple amplified copies of a single nucleic acid molecule—referred to as deep sequencing. The thought on deep sequencing is that if a particular nucleotide of a nucleic acid molecule is sequenced multiple times, then one can more easily identify rare sequence variants or mutations. In fact, however, the amplification and sequencing process has a fixed error rate, so no matter how few or how many times a nucleic acid molecule is sequenced, a person of skill in the art cannot distinguish with certainty a polymerase error artifact from a true mutation.

While being able to sequence many different DNA molecules collectively is advantageous in terms of cost and time, the price for this efficiency and convenience is that various PCR errors complicate mutational analysis as long as their frequency is comparable to that of mutations arising in vivo—in other words, genuine in vivo mutations will be essentially indistinguishable from changes that are artifacts of PCR or sequencing errors.

Thus, the present disclosure, in a further aspect, provides methods for identifying mutations present before amplification or sequencing of a double-stranded nucleic acid library wherein the target molecules include a barcode (i.e., identifier tag) so that sequencing each complementary strand can be connected back to the original molecule. Furthermore, the strand (i.e., sense or antisense) from which a mutation arose can be ascertained in target molecules that include a strand index sequence comprising a non-complementary region flanked by complementary regions capable of forming duplex structures.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. The term “consisting essentially of” limits the scope of a claim to the specified materials or steps, or to those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

A “nucleic acid molecule” or “polynucleotide” refers to a single- or double-stranded linear or circular polynucleotide containing either deoxyribonucleotides or ribonucleotides that are linked by 3′-5′-phosphodiester bonds. A nucleic acid molecule includes DNA molecules, such as genomic, mitochondrial, cDNA, or plasmid DNA molecules.

Variants of the polynucleotides of this disclosure are also contemplated. Variant polynucleotides are at least 80%, 85%, 90%, and preferably 95%, 99%, or 99.9% identical to one of the primers or polynucleotides as described herein, or that hybridizes to one of those primers or polynucleotides of defined sequence under stringent hybridization conditions of 0.015M sodium chloride, 0.0015M sodium citrate at about 65-68° C. or 0.015M sodium chloride, 0.0015M sodium citrate, and 50% formamide at about 42° C. The polynucleotide variants retain the capacity to encode a binding domain or fusion protein thereof having the functionality described herein.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015M sodium chloride, 0.0015M sodium citrate at about 65-68° C. or 0.015M sodium chloride, 0.0015M sodium citrate, and 50% formamide at about 42° C. (see Sambrook et al., Molecular Cloning: A Laboratory Manual, 2nd Ed., Cold Spring Harbor Laboratory, Cold Spring Harbor, N.Y., 1989).

More stringent conditions (such as higher temperature, lower ionic strength, higher formamide, or other denaturing agent) may also be used; however, the rate of hybridization will be affected. In instances wherein hybridization of deoxyoligonucleotides is concerned, additional exemplary stringent hybridization conditions include washing in 6×SSC, 0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides).

The terms “identical” or “percent identity,” in the context of two or more polypeptide or nucleic acid molecule sequences, means two or more sequences or subsequences that are the same or have a specified percentage of amino acid residues or nucleotides that are the same over a specified region (e.g., 60%, 65%, 70%, 75%, 80%, 85%, 90%, 91%, 92%, 93%, 94%, 95%, 96%, 97%, 98%, 99%, or 100% identity), when compared and aligned for maximum correspondence over a comparison window, or designated region, as measured using methods known in the art, such as a sequence comparison algorithm, by manual alignment, or by visual inspection. For example, preferred algorithms suitable for determining percent sequence identity and sequence similarity are the BLAST and BLAST 2.0 algorithms used at default settings, which are described in Altschul et al. (1977) Nucleic Acids Res. 25:3389 and Altschul et al. (1990) J. Mol. Biol. 215:403, respectively.

A “nucleic acid molecule mutation” or “mutation” refers to a change in the nucleotide sequence of a nucleic acid molecule as compared to a reference nucleic acid molecule. A mutation may be caused by radiation, viruses, transposons, mutagenic chemicals, errors that occur during meiosis or DNA replication, or hypermutation. A mutation can result in several different types of sequence changes, including nucleotide substitution, insertion, deletion or any combination thereof.

As used herein, “target nucleic acid molecule” or “target nucleic acid,” and variants thereof, refer to a nucleic acid molecule or fragments thereof that are the subject of a query of mutational status or mutational spectrum as compared to a parent, reference or wild-type target nucleic acid molecule. A target nucleic acid molecule includes genes or fragments thereof, optionally including non-coding sequence(s). Target nucleic acids include fragments from longer molecules, such as genomic or plasmid nucleic acid molecules, which may be generated using a variety of techniques known in the art, such as mechanical shearing or specific cleavage with restriction endonucleases. A target nucleic acid molecule can be incorporated into another nucleic acid molecule, e.g., a target nucleic acid molecule can be inserted into a vector.

As used herein, a “library of double-stranded circular template molecules” refers to a collection or population of double-stranded nucleic acid molecules or fragments thereof, which includes a target nucleic acid molecule. In certain embodiments, the collection or population of double-stranded nucleic acid molecules or fragments thereof, including a target nucleic acid molecule, is incorporated or cloned into a vector and referred to as a “vector library of double-stranded nucleic acid molecules.” If desired, a vector library or library of nucleic acid molecules may be introduced (e.g., transformed, transfected, electroporated) into an appropriate host cell. The target nucleic acid molecules of this disclosure may be introduced into a variety of different vector backbones (such as plasmids, cosmids, viral vectors, or the like) so that the vector library of nucleic acid molecules can self-replicate in, or integrate into the genome of, a host cell of choice (such as bacteria, yeast, insect cells, mammalian cells, or the like). The library of double-stranded nucleic acid molecules, including those that are incorporated into a vector to produce a vector library of double-stranded nucleic acid molecules, may be from a natural source (e.g., genomic DNA), a synthetically produced sample, a recombinantly produced sample (e.g., amplified), or any combination thereof. Prior to insertion into a vector, one or more nucleic acid molecules of interest may be processed to produce a library (plurality) of molecules, such as by mechanical shearing, enzymatic blunting of ends, addition of specific overhangs, specific cleavage with restriction endonuclease(s), or the like, to, for example, improve cloning.

As used herein, a “vector” is a nucleic acid molecule that is capable of transporting another nucleic acid molecule. Vectors may be, for example, plasmids, cosmids, viruses, or phage. An “expression vector” is a vector that is capable of directing the expression of a protein encoded by one or more genes or coding nucleic acid molecules carried by the vector when it is present in the appropriate environment, such as in a host cell.

As used herein, a “nucleic acid molecule primer” or “primer” and variants thereof refers to short nucleic acid sequences that a DNA polymerase can use to begin synthesizing a complementary DNA strand of the template molecule bound by the primer. A primer sequence can vary in length from 5 nucleotides to about 150 nucleotides in length, from about 10 nucleotides to about 35 nucleotides, or are about 15, 16, 17, 18, 19, 20, 21, 22, 23, 24, 25, 26, 27, 28, 29, 30, 31, 32, 33, 34, or 35 nucleotides in length. In certain embodiments, a nucleic acid molecule primer that is complementary to a nucleic acid of interest (e.g., target nucleic acid molecule) can be used to initiate an amplification reaction, a sequencing reaction, or both.

As used herein, a “priming site” and variants thereof are short, known nucleic acid sequences contained in the vector or target nucleic acid molecule. A priming site can vary in length from 5 nucleotides to about 150 nucleotides in length, about 10 nucleotides to about 30 nucleotides in length, or about 15 nucleotides to about 20 nucleotides in length. In certain embodiments, a priming site is included as part of the vector and may be included at one end or part of an adaptor sequence, or included at one end or part of a barcode nucleic acid molecule, or combinations thereof. A nucleic acid molecule primer that is complementary to a priming site included in a library of the present disclosure can be used to initiate an amplification reaction (e.g., PCR), a sequencing reaction, or both.

As used herein, a “library primer” refers to a primer that is capable of hybridizing to a priming site present in each member of a vector library of double-stranded nucleic acid molecule (e.g., DNA). A library primer can prime the amplification of a double-stranded nucleic acid molecule insert or a portion thereof located in a vector library, such as a target nucleic acid molecule, and priming of the amplification reaction (e.g., PCR) can be independent of the sequence of the insert because the library primer is fully complementary or substantially complementary (e.g., 80%, 85%, 90%, 95%, 99% or greater sequence identity) to a sequence in the vector that is upstream or downstream of the double-stranded nucleic acid molecule insert. A library primer may comprise, for example, from about 15 nucleotides to about 150 nucleotides, from about 15 nucleotides to about 50 nucleotides, from about 20 nucleotides to about 40 nucleotides, or comprise about 14, 15, 16, 17, 18, 19, 20, 21, 22, 23, 24, 25, 26, 27, 28, 29, 30, 31, 32, 33, 34, or 35 nucleotides, that have at least about 80% or 90% sequence identity with a first priming site. In some embodiments, a library primer is complementary to and is capable of hybridizing to all or a portion of an adaptor sequence, such as a first adaptor sequence found on a library vector—that is, all or a portion of a first adaptor sequence comprises a first priming site on a vector. It is understood that due to the double-stranded structure of a vector library, a library primer that is complementary to all or a portion of an adaptor sequence may also be said to include or contain the first adaptor sequence. Adaptor sequences are known in the art and are useful in high-throughput or next-generation sequencing methodologies. Exemplary adaptor sequences include Nextera adaptor sequences, Roche 454 adaptor sequences, and Ion Xpress adaptor sequences. A library primer of this disclosure can be a forward primer or a reverse primer, and can be used to initiate an amplification reaction, a sequencing reaction, or both.

As used herein, a “targeting primer” comprises a polynucleotide having a sequence that is capable of hybridizing with a target nucleic acid molecule of interest or a portion thereof, and optionally comprises a further priming site (e.g., an adaptor or portion thereof). In some embodiments, a targeting primer is fully complementary or substantially complementary (e.g., 80%, 85%, 90%, 95%, 99% or greater sequence identity) to a region unique to a target nucleic acid molecule. A targeting primer may comprise, for example, from about 15 nucleotides to about 250 nucleotides, from about 30 nucleotides to about 100 nucleotides, or from about 25 nucleotides to about 50 nucleotides, wherein from 15 to 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides have at least about 80% or 90% sequence identity to the target nucleic acid molecule or portion thereof. In some embodiments, a targeting primer may further comprise a barcode, an index, or any combination thereof. A targeting primer can be a forward primer or a reverse primer, and can be used to initiate an amplification reaction, a sequencing reaction, or both.

As used herein, a “nested primer” refers to a polynucleotide having a sequence that is capable of hybridizing to a target nucleic acid molecule and does not overlap with the target region of a targeting primer or the 5′-end of the nested primer may partially overlap with the target region of the targeting primer. A nested primer may be used in a successive round of polymerase chain reaction (“nested PCR”), following a first round of PCR using the targeting primer, resulting in an amplicon that is a fragment of the template amplicon. Use of a nested PCR can increase the specificity, sensitivity or both of PCR. A nested primer optionally comprises a further priming site (e.g., an adaptor or portion thereof). A nested primer may comprise, for example, from about 15 nucleotides to about 250 nucleotides, from about 30 nucleotides to about 100 nucleotides, or from about 25 nucleotides to about 50 nucleotides, wherein from 15 to 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides have at least about 80% or 90% sequence identity to the target nucleic acid molecule or portion thereof. In some embodiments, a nested primer may further comprise a barcode, an index, or both. A nested primer can be a forward primer or a reverse primer, and can be used to initiate an amplification reaction, a sequencing reaction, or both.

As used herein, a “non-targeting primer” refers to a polynucleotide having a sequence that is capable of hybridizing to a priming site adjacent to or near the double-stranded template nucleic acid molecule insert that is on the opposite end from an adaptor and barcode linked to the template nucleic acid molecule. A non-targeting primer optionally comprises a further priming site (e.g., an adaptor or portion thereof). In certain embodiments, the non-targeting primer hybridizes to a priming site that is contained wholly or partially within the vector backbone. In some embodiments, a targeting primer is fully complementary or substantially complementary (e.g., about 80%, 85%, 90%, 95%, 99% or greater sequence identity) to a region within the vector backbone. A non-targeting primer may comprise, for example, from about 15 nucleotides to about 250 nucleotides, from about 30 nucleotides to about 100 nucleotides, or from about 25 nucleotides to about 50 nucleotides, wherein from 15 to 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides have at least 80% sequence identity to the vector backbone. In some embodiments, a non-targeting primer may further comprise a barcode, an index, or both. A non-targeting primer can be a forward primer or a reverse primer, and can be used to initiate an amplification reaction, a sequencing reaction, or both.

The term “polymerase chain reaction (PCR)” as used herein refers to a method for amplifying a single or few copies of a nucleic acid molecule across several orders of magnitude. PCR relies on thermal cycling, comprising cycles of repeated heating and cooling of the reaction for nucleic acid melting and enzymatic replication of the DNA. Primers contain sequences complementary to regions flanking or within (partially or fully) a target nucleic acid molecule, and such template nucleic acid molecules are replicated by a thermostable polymerase enzyme (e.g., Taq polymerase). As PCR progresses, the DNA generated is itself used as a template for replication, setting in motion a chain reaction in which the DNA template is exponentially amplified. PCR can be extensively modified to perform a wide array of genetic manipulations and analyses. Exemplary methods of performing PCR are described in U.S. Pat. Nos. 4,683,195; 4,683,202; 4,965,188; 4,889,818; 5,079,352; 5,038,852; 5,612,473; 5,602,756; 5,538,871; and 5,475,610, which methods are herein incorporated by reference in their entirety.

As used herein, “nucleic acid sequencing” means the determination of the order of nucleotides in a nucleic acid molecule or a sample of nucleic acid molecules, wherein the nucleic acid molecules include DNA, cDNA or RNA molecules.

As used herein, “next generation sequencing” refers to high-throughput sequencing methods that allow the sequencing of thousands or millions of molecules in parallel. Examples of next generation sequencing methods include sequencing by synthesis, sequencing by ligation, sequencing by hybridization, polony sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by 454 Life Sciences, Agencourt Bioscience, Applied Biosystems, LI-COR Biosciences, Microchip Biotechnologies, Network Biosystems, NimbleGen Systems, Illumina, and VisiGen Biotechnologies, including formats such as parallel bead arrays, sequencing by synthesis, sequencing by ligation, capillary electrophoresis, electronic microchips, “biochips,” microarrays, parallel microchips, and single-molecule arrays, as reviewed by Service (Science 311:1544-1546, 2006).

As used herein, “single molecule sequencing” or “third generation sequencing” refers to high-throughput sequencing methods wherein reads from single molecule sequencing instruments represent sequencing of a single molecule of DNA. Unlike next generation sequencing methods that rely on PCR to grow clusters of a given DNA template, attaching the clusters of DNA templates to a solid surface that is then imaged as the clusters are sequenced by synthesis in a phased approach, single molecule sequencing interrogates single molecules of DNA and does not require PCR amplification or synchronization. Single molecule sequencing includes methods that need to pause the sequencing reaction after each base incorporation (‘wash-and-scan’ cycle) and methods which do not need to halt between read steps. Examples of single molecule sequencing methods include single molecule real-time sequencing, nanopore-based sequencing, and direct imaging of DNA using advanced microscopy.

As used herein, “base calling” refers to the computational conversion of raw or processed data from a sequencing instrument into quality scores and then actual sequences. For example, many of the sequencing platforms use optical detection and charge coupled device (CCD) cameras to generate images of intensity information (i.e., intensity information indicates which nucleotide is in which position of a nucleic acid molecule), so base calling generally refers to the computational image analysis that converts intensity data into sequences and quality scores. Another example is the ion torrent sequencing technology, which employs a proprietary semiconductor ion sensing technology to detect release of hydrogen ions during incorporation of nucleotide bases in sequencing reactions that take place in a high density array of micro-machined wells. There are other examples of methods known in the art that may be employed for simultaneous sequencing of large numbers of nucleotide molecules. Various base calling methods are described in, for example, Niedringhaus et al. (Anal. Chem. 83:4327, 2011), the methods of which are herein incorporated by reference.

As used herein, “strand index sequence” refers to two polynucleotide molecules capable of forming double-stranded regions that flank a non-complementary region. The 5′- and 3′-ends of the first and second strands of the strand index sequence each comprise a complementary region capable of forming stable duplex structure, wherein the non-complementary region is is disposed between duplex structures. When the ends of the two polynucleotide molecules are duplexed, the non-complementary region disposed between the double-stranded structures forms a “bubble” in which the two strands are unpaired. The length of the non-complementary region should be long enough to provide a unique, strand-specific identifier (e.g., at least three nucleotides) and short enough as to not reduce the overall stability of the flanking duplex regions. The non-complementary region may comprise, for example, from about 2 to about 30 nucleotides, from about 2 to about 20 nucleotides, from about 2 to about 10 nucleotides, from about 2 to about 5 nucleotides, from about 3 to about 30 nucleotides, from about 3 to about 20 nucleotides, from about 3 to about 20 nucleotides, from about 3 to about 10 nucleotides, or from about 3 to about 5 nucleotides. In certain embodiments, the non-complementary sequence has 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. Each complementary region capable of forming a duplex structure may comprise, for example, from about 10 to about 50 nucleotides, from about 10 to about 40 nucleotides, from about 10 to about 30 nucleotides, from about 10 to about 20 nucleotides, or from about 10 to about 15 nucleotides. In certain embodiments, the complementary region capable of forming a duplex structure contains about 10, 11, 12, 13, 14, 15, 16, 17, 18, 19, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides, wherein the nucleotide sequence on the sense strand is complementary to the antisense strand. While inclusion of a double-stranded bar code in the vector library of double-stranded nucleic acid molecules allows for each sequence read to be traced back to the original double-stranded nucleic acid molecule, inclusion of a strand index sequence in the vector library further permits each sequence read to be traced back to a particular strand (i.e., sense or antisense).

As used herein, “read family” refers to sequence reads containing the same bar code and originating from the same nucleic acid molecule.

As used herein, a “consensus sequence” when used in reference to a read family refers to a common sequence derived from the reads in a family. In certain embodiments, a read family has at least three members before a consensus sequence is determined. Sequencing errors are identified by comparing reads within a read family and removed to create an error-corrected consensus sequence.

In the following description, certain specific details are set forth in order to provide a thorough understanding of various embodiments of this disclosure. However, upon reviewing this disclosure, one skilled in the art will understand that the invention may be practiced without many of these details. In other instances, newly emerging next generation sequencing technologies, as well as well-known or widely available next generation sequencing methods (e.g., chain-termination sequencing, dye-terminator sequencing, reversible dye-terminator sequencing, sequencing by synthesis, sequencing by ligation, sequencing by hybridization, polony sequencing, pyrosequencing, ion semiconductor sequencing, nanoball sequencing, nanopore sequencing, single molecule sequencing, FRET sequencing, base-heavy sequencing, and microfluidic sequencing), have not all been described in detail to avoid unnecessarily obscuring the descriptions of the embodiments of the present disclosure. Descriptions of some of these methods can be found, for example, in PCT Publication Nos. WO 98/44151, WO 00/18957, and WO 2006/08413; and U.S. Pat. Nos. 6,143,496, 6,833,246, and 7,754,429; and U.S. Patent Application Publication Nos. U.S. 2010/0227329 and U.S. 2009/0099041.

Various embodiments of the present disclosure are described for purposes of illustration, in the context of use with vectors containing a library of nucleic acid fragments (e.g., genomic or cDNA library). However, as those skilled in the art will appreciate upon reviewing this disclosure, use with other nucleic acid libraries or methods for making a library of nucleic acid fragments may also be suitable.

In certain aspects, provided herein are methods for detecting or identifying mutations in a nucleic acid molecule, comprising amplifying a template target nucleic acid molecule from a vector library of double-stranded template nucleic acid molecules with a plurality of primers comprising library primer and a targeting primer, wherein each vector comprises a first adaptor and a barcode adjacent to or near the double-stranded template nucleic acid molecule insert and at least one vector of the library comprises a template target nucleic acid molecule; and wherein the library primer comprises a sequence capable of hybridizing to a first priming site within or near the first adaptor and the targeting primer comprises a sequence capable of hybridizing to a target region within the target nucleic acid molecule; thereby producing an amplified target nucleic acid molecule comprising a first adaptor region and a barcode adjacent to a target nucleic acid molecule, which amplified target nucleic acid molecule can be sequenced using the library and targeting primers to identify or detect mutations found in the template target nucleic acid molecule.

In further aspects, provided herein are methods for detecting or identifying mutations in a nucleic acid molecule, comprising sequencing with a plurality of primers comprising a library primer and a targeting primer, a template target nucleic acid molecule amplified from a vector library of double-stranded template nucleic acid molecules, wherein each vector comprises a first adaptor and a barcode adjacent to or near the double-stranded template nucleic acid molecule insert and at least one vector of the library comprises a template target nucleic acid molecule; and wherein the amplified target nucleic acid molecule comprising a first adaptor region and a barcode adjacent to a target nucleic acid molecule is produced using the library primer and the targeting primer, wherein the library primer comprises a sequence capable of hybridizing to a first priming site within or near the first adaptor and the targeting primer comprises a sequence capable of hybridizing to a target region within the target nucleic acid molecule; thereby identifying or detecting mutations found in the template target nucleic acid molecule.

In other aspects, provided herein are methods for detecting or identifying mutations in a nucleic acid molecule, comprising amplifying a template target nucleic acid molecule from a vector library of double-stranded template nucleic acid molecules with a plurality of primers comprising a library primer and a non-targeting primer, wherein each vector comprises a first adaptor and a barcode adjacent to or near the double-stranded template nucleic acid molecule insert and at least one vector of the library comprises a template target nucleic acid molecule; and wherein the library primer comprises a sequence capable of hybridizing to a first priming site within or near the first adaptor and the non-targeting primer comprises a sequence capable of hybridizing to a second priming site adjacent to the double-stranded template nucleic acid molecule insert and on the opposite end from the first adaptor and the bar code; thereby producing an amplified target nucleic acid molecule comprising a first adaptor region and a barcode adjacent a target nucleic acid molecule, which amplified target nucleic acid molecule can be sequenced using the library and non-targeting primers to identify or detect mutations found in the template target nucleic acid molecule.

As used herein, “adjacent to” or “near” refers to the proximity of a double-stranded template nucleic acid molecule insert in a vector to other elements in the vector, such as an adaptor, barcode, or priming site, which includes abutting against a vector element (e.g., no nucleotides separate the insert and vector element); or a separation of one to about 100 nucleotides. In certain embodiments, a double-stranded template nucleic acid molecule is adjacent to or near a barcode or adaptor when there is a separation of a number of nucleotides that remain after a cut by a restriction endonuclease, which can range from one nucleotide to about 33 nucleotides, one nucleotide to about 30 nucleotides, one nucleotide to about 25 nucleotides, one nucleotide to about 20 nucleotides, or one nucleotide to about 10 nucleotides (e.g., cleavage with XcmI results in 7 to 8 nucleotides between the insert and the vector element, such as a barcode). In some embodiments, a double-stranded template nucleic acid molecule is adjacent to or near a barcode or adaptor when there is a separation of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 nucleotides that remain after a cut by a restriction endonuclease. In certain embodiments, a double-stranded template nucleic acid molecule is adjacent to or near a second priming site of a non-targeting primer when there is separation of a number of nucleotides that remain after a cut by a restriction endonuclease, which can range from 0, 1, 2, 3, 4, 5, 6, 7, or 8 to about 100 nucleotides, 0, 1, 2, 3, 4, 5, 6, 7, or 8 to about 90 nucleotides, 0, 1, 2, 3, 4, 5, 6, 7, or 8 to about 80 nucleotides, 0, 1, 2, 3, 4, 5, 6, 7, or 8 to about 70 nucleotides, 0, 1, 2, 3, 4, 5, 6, 7, 8 to about 60 nucleotides, 0, 1, 2, 3, 4, 5, 6, 7, or 8 to about 50 nucleotides, 0, 1, 2, 3, 4, 5, 6, 7, or 8 to about 40 nucleotides, 0, 1, 2, 3, 4, 5, 6, 7, or 8 to about 30 nucleotides, or 0, 1, 2, 3, 4, 5, 6, 7, or 8 to about 20 nucleotides.

In some embodiments, the library of double-stranded nucleic acid molecules comprises genomic DNA, mitochondrial DNA, cDNA, plasmid DNA, or a combination thereof. In certain embodiments, the library of double-stranded nucleic acid molecules comprises genomic DNA. In certain embodiments, a nucleic acid molecule is genomic DNA. In some embodiments, a nucleic acid molecule is mitochondrial DNA. In some embodiments, the nucleic acid molecule is a cDNA. In some embodiments, the nucleic acid molecule is a plasmid DNA. A target nucleic acid molecule is any nucleic acid molecule of interest, including genomic DNA, mitochondrial DNA, cDNA, or plasmid DNA, in which detection of a sequence or mutation is desirable (e.g., a gene encoding 16S rRNA or an oncogene). A reference target nucleic acid molecule sequence is a wild type or normal sequence of a selected target nucleic acid molecule. A target nucleic acid molecule may have more than one reference sequence. Methods for isolating nucleic acid molecules for use in the methods described herein are well known in the art.

The disclosed methods may be used to detect any mutation within a target nucleic acid molecule. In some embodiments, a mutation is a deletion of one or more nucleotides. In some embodiments, a mutation is an insertion or substitution of one or more nucleotides. In some embodiments, a mutation includes rearrangements of large segments of nucleotides, such as chromosomal translocations, inversions, or duplications.

As used herein, an “adaptor,” “adaptor region” or “adaptor sequence” refers to a nucleic acid molecule of known composition, generally ranging in length from about 20 to about 150 nucleotides, which are designed to facilitate compatibility with next generation sequencing or high-throughput sequencing methods (e.g., Illumina sequencing technology). In some embodiments, a first adaptor is located upstream of a 5′-barcode or downstream of a 3′-barcode. In some embodiments, adaptors contain sequences useful for amplification and sequencing, or other processing of the target nucleic acid molecules following amplification. In certain embodiments, adaptor sequences contain restriction endonuclease sites or primer sites for bridge amplification (e.g., “a flow cell adaptor sequence”), PCR amplification, sequencing (e.g., “read primer” which is used to prime a sequencing reaction), or a combination thereof. In certain embodiments, an adaptor sequence may include a unique index sequence specific for a particular sample so that a library can be pooled with other libraries having different index sequences to facilitate multiplex sequencing (also referred to as “multiplexing”). In certain embodiments, adaptors may be used to tag a nucleic acid with a DNA tag, to provide sequences that enable hybridization for the purposes of capture, or to add chemically modified nucleic acid sequences such as biotin-containing adaptors.

In certain embodiments, the first adaptor region is located 5′ of the barcode and the barcode is located 5′ of the target nucleic acid. In other embodiments, the first adaptor region is located 3′ of the barcode and the barcode is located 3′ of the target nucleic acid. In some embodiments, the first adaptor and second adaptor regions are adaptors compatible with systems known in the art such as those provided by Illumina, Roche 454, Ion Torrent, or SOLiD Colorspace or fragments of such adaptors. In some embodiments, the first and second adaptor regions are Nextera adaptor sequences or portions thereof. For example, adaptor sequences as set forth in SEQ ID NO:11 and SEQ ID NO:12 are exemplary Nextera flow cell adaptor sequences that anneal to complementary oligonucleotides on flow cell surfaces. Flow cell adaptor sequences allow cluster generation (bridge amplification) on the flow cell surface. Fragments of commercially available adaptors that may be used in the methods disclosed herein may possess sufficient sequences for priming PCR amplification, bridge amplification, sequencing, or any combination thereof, and optionally, may not contain other sequences, for example transposon adaptor sequences (i.e., for Nextera adaptors). In certain embodiments, a first adaptor sequence comprises a flow adaptor sequence, an index sequence, and a read primer sequence. In certain embodiments, a first adaptor sequence consists or comprises a sequence selected from the following pairs of sequences SEQ ID NOS:9 and 10; SEQ ID NOS: 11 and 12; SEQ ID NOS: 13 and 14; and SEQ ID NOS: 15 and 16. It is understood by a person of skill in the art that a first adaptor may be either adaptor sequence of an adaptor pair, and the second adaptor may be selected from the remaining adaptor sequence of the pair.

The library priming site (first priming site) is located in the double-stranded circular template nucleic acid molecule such that a complementary primer can prime the amplification of the adaptor region, barcode, and target nucleic acid. In other words, the library priming site sequence is located on the opposite side of the barcode as the target nucleic acid molecule. Thus, if the barcode is located 5′ of the target nucleic acid molecule, the library priming site sequence is 5′ of the barcode. If the barcode is located 3′ of the target nucleic acid molecule, the library priming site sequence is 3′ of the barcode. For example, if read in a 5′ to 3′ direction, the orientation can be 5′-first priming site-first adaptor-barcode-target nucleic acid molecule-3′. Alternately, the orientation can be 5′-target nucleic acid molecule-barcode-first adaptor-first priming site-3′. A library primer comprises a sequence capable of hybridizing to a first priming site within or near the first adaptor and may contain bases that are complementary to the first adaptor or to the first adaptor and a portion of the vector backbone. A library primer may also possess additional sequence (e.g., at its 5′-end) that is not complementary to either the first adaptor or the vector backbone. In some embodiments, the first adaptor region comprises all or a portion of the first priming site. In some embodiments, the first priming site comprises all or a portion of the first adaptor region. In certain embodiments, the first priming site comprises a Nextera adaptor sequence. In certain embodiments where first adaptor sequence comprises SEQ ID NO:9 or SEQ ID NO:11, the first priming site corresponds to a nucleic acid molecule having the sequence of SEQ ID NO:2. In other embodiments wherein the first adaptor sequence comprises SEQ ID NO:10 or SEQ ID NO:12, the first priming site corresponds to a nucleic acid molecule having the sequence of SEQ ID NO:4. A nucleic acid molecule primer that is complementary to the first priming site included in a library of the present disclosure can be used to initiate a PCR amplification reaction, a sequencing reaction, or both.

In some embodiments, a library primer sequence can vary in length from 15 nucleotides to about 150 nucleotides in length having at least about 80% or 90% complementary to a first priming site, from about 10 nucleotides to about 35 nucleotides, and preferably are about 15, 16, 17, 18, 19, 20, 21, 22, 23, 24, 25, 26, 27, 28, 29, 30, 31, 32, 33, 34, or 35 nucleotides in length that have at least about 80% or 90% complementary to a first priming site. In some embodiments, a library primer comprises all or a portion of an adaptor sequence, or a sequence complementary thereto, and can be used to initiate an amplification reaction, a sequencing reaction, or both. A library primer can be a forward primer or a reverse primer. In some embodiments, a library primer corresponds to a Nextera adaptor, Ion Xpress adaptor, or a Roche 454 Primer A or Primer B sequence. In some embodiments, a first adaptor sequence comprises SEQ ID NO:9 or SEQ ID NO:11, and a library primer has a nucleic acid sequence that corresponds to SEQ ID NO: 2. In other embodiments, a first adaptor sequence comprises SEQ ID NO:10 or SEQ ID NO:12, and a library primer has a nucleic acid sequence that corresponds to SEQ ID NO:4. In other embodiments, a first adaptor sequence comprises SEQ ID NO:13 and a library primer comprises SEQ ID NO:13. In yet other embodiments, a first adaptor sequence comprises SEQ ID NO:14, and a library primer comprises SEQ ID NO:14. In yet other embodiments, a first adaptor sequence comprises SEQ ID NO:15, and a library primer comprises SEQ ID NO:15. In other embodiments, a first adaptor sequence comprises SEQ ID NO:16, and a library primer comprises SEQ ID NO:16.

A targeting primer specific for a first target nucleic acid molecule may be designed to amplify a selected region within a nucleic acid molecule (e.g., a mutational hot spot) or multiple regions within a nucleic acid molecule, or designed to amplify an entire nucleic molecule. A targeting primer specific for a first target nucleic acid molecule may be spaced from about 30, 40, 50, 60, 70, 80, 90, 100, 200, 300, 400, 500, 600, 700, 800, 900, 1,000, 1,500, or 2,000 nucleotides apart from the library primer on the same strand of the vector including the first target nucleic acid molecule. In certain embodiments, the targeting primer and the library primer may be spaced from about 50 to about 1,000 nucleotides apart on the same strand the nucleic acid molecule. By utilizing a plurality of primers designed with selective positioning and spacing, entire nucleic acid molecules (e.g., genes, transcripts, genomes) may be interrogated in a single assay. In addition, primers designed with selective positioning and spacing can be used to selectively sequence only desired segments of a particular gene or select group of genes.

In some instances it may be advantageous to include non-standard nucleotides or modified internucleotide linkages in a primer (e.g., to enhance specificity, reduce degradation). Accordingly, in certain embodiments, a library primer or a targeting primer comprises one or more locked nucleic acid (LNA) molecule, peptide nucleic acid (PNA) molecule, morpholino subunit, universal-binding nucleotide (such as C-phenyl, C-naphthyl, inosine, azole carboxamide, 1-β-D-ribofuranosyl-4-nitroindole, 1-β-D-ribofuranosyl-5-nitroindole, 1-β-D-ribofuranosyl-6-nitroindole, 1-β-D-ribofuranosyl-3-nitropyrrole), 2′-sugar substitution such as a 2′-O-methyl, 2′-O-methoxyethyl, 2′-O-2-methoxyethyl, 2′-O-allyl, or halogen like 2′-fluoro), modified internucleotide linkages (such as phosphorothioate, chiral phosphorothioate, phosphorodithioate, phosphotriester, aminoalkylphosphotriester, methyl phosphonate, alkyl phosphonate, 3′-alkylene phosphonate, 5′-alkylene phosphonate, chiral phosphonate, phosphonoacetate, thiophosphonoacetate, phosphinate, phosphoramidate, 3′-amino phosphoramidate, aminoalkylphosphoramidate, selenophosphate, thionophosphoramidate, thionoalkylphosphonate, thionoalkylphosphotriester, or boranophosphate linkage), or any combination thereof.

In certain embodiments, a targeting primer specific for a first target nucleic acid molecule comprises a second adaptor sequence, or a portion thereof. The second adaptor should be compatible with the same sequencing system in which the first adaptor sequence can function. For example, if the first adaptor sequence is compatible with an Illumina sequencing system, then the second adaptor sequence should also be compatible with the Illumina sequencing system. In certain embodiments, a second adaptor sequence comprises sufficient sequence to prime a PCR amplification reaction, bridge amplification reaction, sequencing reaction, or any combination thereof, and optionally, may lack other additional sequences, such as a transposon adaptor sequence (i.e., as found in some Nextera adaptors). In some embodiments, a second adaptor sequence comprises a flow adaptor sequence, an index sequence, and a read primer sequence. In some embodiments, a second adaptor sequence or portion thereof is located at the 5′-end of the targeting primer. In some embodiments, the second adaptor sequence is a Nextera adaptor sequence. In certain embodiments, a first adaptor is a nucleic acid molecule having the sequence of SEQ ID NO:2, and a second adaptor is a nucleic acid molecule having the sequence of SEQ ID NO: 4, or vice versa. In certain embodiments, if a first adaptor is a nucleic acid molecule having the sequence of SEQ ID NO:9, a second adaptor is a nucleic acid molecule having the sequence of SEQ ID NO:10, or vice versa. In other embodiments, if a first adaptor is a nucleic acid molecule having the sequence of SEQ ID NO:11, a second adaptor is a nucleic acid molecule having the sequence of SEQ ID NO:10, or vice versa. In other embodiments, if a first adaptor is a nucleic acid molecule having the sequence of SEQ ID NO:9, a second adaptor is a nucleic acid molecule having the sequence of SEQ ID NO:12, or vice versa. In other embodiments, if a first adaptor is a nucleic acid molecule having the sequence of SEQ ID NO:11, a second adaptor is a nucleic acid molecule having the sequence of SEQ ID NO:12, or vice versa. In yet other embodiments, if a first adaptor is a nucleic acid molecule having the sequence of SEQ ID NO:13, a second adaptor is a nucleic acid molecule having the sequence of SEQ ID NO:14, or vice versa. In yet other embodiments, if a first adaptor is a nucleic acid molecule having the sequence of SEQ ID NO:15, a second adaptor is a nucleic acid molecule having the sequence of SEQ ID NO:16, or vice versa.

In certain embodiments, a non-targeting primer may be used instead of a targeting primer with the library primer. A non-targeting primer comprises a sequence capable of hybridizing to a second priming site adjacent to the double-stranded template nucleic acid molecule insert, which priming site is on the opposite end from the first adaptor and the barcode. In certain embodiments, the vector backbone comprises all or a portion of the second priming site of the non-targeting primer. Thus, if a library primer is located 5′ to the double-stranded template nucleic acid molecule insert, the non-targeting primer is located 3′ to the double-stranded template nucleic acid molecule insert. If a library primer is located 3′ to the double-stranded template nucleic acid molecule insert, the non-targeting primer is located 5′ to the double-stranded template nucleic acid molecule insert. For example, if read in a 5′ to 3′ direction, the orientation can be 5′-first priming site-first adaptor-barcode-target nucleic acid molecule-second priming site-3′. Alternately, the orientation can be 5′-second priming site-target nucleic acid molecule-barcode-first adaptor-first priming site-3′.

In some embodiments, a non-targeting primer sequence can vary in length from 15 nucleotides to about 150 nucleotides in length having at least 80% complementary to a second priming site, from about 10 nucleotides to about 35 nucleotides, and preferably are about 15, 16, 17, 18, 19, 20, 21, 22, 23, 24, 25, 26, 27, 28, 29, 30, 31, 32, 33, 34, or 35 nucleotides in length that have at least 80% complementary to a second priming site. A non-targeting primer can be a forward primer or a reverse primer.

In certain embodiments, a second priming site of a non-targeting primer is adjacent to or near a double-stranded template nucleic acid molecule when there is separation of a number of nucleotides that remain after a cut by a restriction endonuclease, which can range from one to 200 nucleotides (e.g., the 3′-end of the non-targeting primer is spaced 1 to 200 nucleotides from the double-stranded template nucleic acid molecule). In some embodiments, the 3′-end of the non-targeting primer is spaced 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides from the double-stranded template nucleic acid molecule.

In certain embodiments, the non-targeting primer comprises a second adaptor sequence or portion thereof. In some embodiments, the non-targeting primer comprises a second adaptor sequence or portion thereof on its 5′-end. As apparent to a person of ordinary skill in the art, the second adaptor provides compatibility with the same sequencing system as the first adaptor sequence. For example, if the first adaptor sequence is compatible with an Illumina sequencing system, then the second adaptor sequence is also compatible with the Illumina sequencing system. In certain embodiments, a second adaptor sequence comprises sufficient sequence to prime a PCR amplification reaction, bridge amplification reaction, sequencing reaction, or any combination thereof, and optionally, may lack other additional sequences, such as a transposon adaptor sequence (i.e., as found in some Nextera adaptors). In some embodiments, a second adaptor sequence comprises a flow adaptor sequence, an index sequence, and a read primer sequence. In further embodiments, the second adaptor sequence is a Nextera adaptor sequence, Ion Xpress adaptor sequence, or a Roche 454 adaptor sequence. In certain embodiments, if a first adaptor is a nucleic acid molecule having the sequence of SEQ ID NO:9, a second adaptor is a nucleic acid molecule having the sequence of SEQ ID NO:10, or vice versa. In other embodiments, if a first adaptor is a nucleic acid molecule having the sequence of SEQ ID NO:11, a second adaptor is a nucleic acid molecule having the sequence of SEQ ID NO:10, or vice versa. In other embodiments, if a first adaptor is a nucleic acid molecule having the sequence of SEQ ID NO:9, a second adaptor is a nucleic acid molecule having the sequence of SEQ ID NO:12, or vice versa. In other embodiments, if a first adaptor is a nucleic acid molecule having the sequence of SEQ ID NO:11, a second adaptor is a nucleic acid molecule having the sequence of SEQ ID NO:12, or vice versa. In yet other embodiments, if a first adaptor is a nucleic acid molecule having the sequence of SEQ ID NO:13, a second adaptor is a nucleic acid molecule having the sequence of SEQ ID NO:14, or vice versa. In yet other embodiments, if a first adaptor is a nucleic acid molecule having the sequence of SEQ ID NO:15, a second adaptor is a nucleic acid molecule having the sequence of SEQ ID NO:16, or vice versa. A non-targeting primer that is complementary to the second priming site included in a library of the present disclosure can be used to initiate a PCR amplification reaction, a sequencing reaction, or both.

A non-targeting primer specific for a second priming site is designed to amplify an entire nucleic molecule. A non-targeting primer specific for a second priming site may be spaced from about 30, 40, 50, 60, 70, 80, 90, 100, 200, 300, 400, 500, 600, 700, 800, 900, 1,000, 1,500, 2,000, 2,500, 3,000, 3,500, 4,000, 4,500, 5,000 nucleotides apart from the library primer on the same strand of the vector including the nucleic acid molecule. In certain embodiments, the non-targeting primer and the library primer may be spaced from about 100 to about 1,500 nucleotides apart on the same strand the nucleic acid molecule. By utilizing a library primer and a non-targeting primer complementary to the vector backbone and flanking the double-stranded nucleic acid molecule insert, all of the nucleic acid molecule inserts represented in a vector library may be amplified. The entire vector library may be sequenced. Alternatively, an enriched vector library may be used as a template for a subsequent round of amplification with a library specific primer and a targeting-primer.

In some instances, it may be advantageous to use a nested PCR approach to enrich a target nucleic acid molecule in a vector library of double-stranded template nucleic acid molecules. Nested PCR can increase the sensitivity and specificity of detection of the target nucleic acid molecule. In a nested PCR approach, a targeting primer is used in combination with a library primer to amplify a target nucleic acid molecule in an initial round of PCR. In certain embodiments, the targeting primer does not include a second adaptor sequence. The amplification products are used as template molecules for a subsequent round of nested PCR using the library primer and a nested primer. A nested primer is a polynucleotide having a sequence that hybridizes with a target nucleic acid molecule and does not overlap with the target region of the targeting primer or the 5′ end of the nested primer may partially overlap with the target region of the targeting primer. Thus, the priming site of the nested primer is internal to the targeting primer. Nested PCR results in an amplified target nucleic acid molecule that is a portion of the template amplicon.

A nested primer may comprise, for example, from about 15 nucleotides to about 250 nucleotides, from about 30 nucleotides to about 100 nucleotides, or from about 25 nucleotides to about 50 nucleotides, wherein from 15 to 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides have at least 80% or 90% sequence identity to the target nucleic acid molecule or portion thereof. A nested primer can be a forward primer or a reverse primer.

In certain embodiments, a nested primer comprises a second adaptor sequence or portion thereof. In some embodiments, a nested primer comprises a second adaptor sequence or portion thereof on its 5′-end. In certain embodiments, a second adaptor sequence comprises sufficient sequence to prime a PCR amplification reaction, bridge amplification reaction, sequencing reaction, or any combination thereof, and optionally, may lack other additional sequences, such as a transposon adaptor sequence (i.e., as found in some Nextera adaptors). In some embodiments, a second adaptor sequence comprises a flow adaptor sequence, an index sequence, a read primer sequence, or any combination thereof.

In further embodiments, the second adaptor sequence is a Nextera adaptor sequence, Ion Xpress adaptor sequence, or a Roche 454 adaptor sequence. In certain embodiments, if a first adaptor is a nucleic acid molecule having the sequence of SEQ ID NO:9, a second adaptor is a nucleic acid molecule having the sequence of SEQ ID NO:10, or vice versa. In other embodiments, if a first adaptor is a nucleic acid molecule having the sequence of SEQ ID NO:11, a second adaptor is a nucleic acid molecule having the sequence of SEQ ID NO:10, or vice versa. In other embodiments, if a first adaptor is a nucleic acid molecule having the sequence of SEQ ID NO:9, a second adaptor is a nucleic acid molecule having the sequence of SEQ ID NO:12, or vice versa. In other embodiments, if a first adaptor is a nucleic acid molecule having the sequence of SEQ ID NO:11, a second adaptor is a nucleic acid molecule having the sequence of SEQ ID NO:12, or vice versa. In yet other embodiments, if a first adaptor is a nucleic acid molecule having the sequence of SEQ ID NO:13, a second adaptor is a nucleic acid molecule having the sequence of SEQ ID NO:14, or vice versa. In yet other embodiments, if a first adaptor is a nucleic acid molecule having the sequence of SEQ ID NO:15, a second adaptor is a nucleic acid molecule having the sequence of SEQ ID NO:16, or vice versa. In some embodiments, a nested primer may further comprise a barcode, an index, or any combination thereof. A nested primer that is complementary to the target nucleic acid molecule can be used to initiate a PCR amplification reaction, a sequencing reaction, or both.

As will be understood by one of ordinary skill in the art, multiple rounds of PCR may be used to add or extend the first or second adaptor sequence flanking a target nucleic acid molecule. For example, a primer (e.g., nested primer, library primer, non-targeting primer, or targeting primer) may comprise a portion of an adaptor (e.g., a truncated adaptor), such as a read primer sequence. Following amplification with the primer comprising a truncated adaptor, a subsequent round of PCR may be performed using a second primer, wherein the second primer comprises the truncated adaptor, or a fragment thereof, and further adaptor components (e.g., an index sequence, a read primer or both) (see, e.g., Example 3).

Accordingly, an exemplary embodiment comprises performing a PCR reaction with a library of genomic DNA molecules using a library primer that comprises a first Nextera adaptor sequence and a targeting primer that targets a specific gene or gene segment of interest, wherein the targeting primer comprises a target specific sequence and a second Nextera adaptor sequence. In certain embodiments, the method comprises performing a PCR reaction with a library of genomic DNA molecules using a library primer that is a first Nextera adaptor sequence and a targeting primer that targets a specific gene or gene segment of interest, wherein the targeting primer comprises a target specific sequence and a second Nextera adaptor sequence. In some embodiments, the first adaptor comprises a polynucleotide sequence of SEQ ID NO:9 or SEQ ID NO:11, the library primer comprises a polynucleotide sequence of SEQ ID NO: 2, the second adaptor sequence comprises a polynucleotide sequence of SEQ ID NO:10 or SEQ ID NO:12, and the targeting primer comprises a target specific sequence and a polynucleotide sequence of SEQ ID NO: 4. In other embodiments, the first adaptor comprises a polynucleotide sequence of SEQ ID NO:10 or SEQ ID NO:12, the library primer comprises a polynucleotide sequence of SEQ ID NO: 4, the second adaptor sequence comprises a polynucleotide sequence of SEQ ID NO:9 or SEQ ID NO:11, and the targeting primer comprises a target specific sequence and a polynucleotide sequence of SEQ ID NO: 2.

Another exemplary embodiment comprises performing a PCR reaction with a library of genomic DNA molecules using a library primer that comprises a first Nextera adaptor sequence and a targeting primer that targets a specific gene or gene segment of interest, wherein the targeting primer comprises a target specific sequence. In certain embodiments, the method comprises performing a PCR reaction with a library of genomic DNA molecules using a library primer that is the first Nextera adaptor sequence and a targeting primer that targets a specific gene or gene segment of interest, wherein the targeting primer comprises a target specific sequence. The amplification products are used as template for a subsequent PCR reaction using the library primer that comprises the first Nextera adaptor sequence and a nested primer comprising a target specific sequence and a second Nextera adaptor sequence. In some embodiments, the first adaptor comprises a polynucleotide sequence of SEQ ID NO:9 or SEQ ID NO:11, the library primer comprises a polynucleotide sequence of SEQ ID NO: 2, the second adaptor sequence comprises a polynucleotide sequence of SEQ ID NO:10 or SEQ ID NO:12, and the targeting primer comprises a target specific sequence and a polynucleotide sequence of SEQ ID NO: 4. In other embodiments, the first adaptor comprises a polynucleotide sequence of SEQ ID NO:10 or SEQ ID NO:12, the library primer comprises a polynucleotide sequence of SEQ ID NO: 4, the second adaptor sequence comprises a polynucleotide sequence of SEQ ID NO:9 or SEQ ID NO:11, and the targeting primer comprises a target specific sequence and a polynucleotide sequence of SEQ ID NO: 2.

Yet another exemplary embodiment comprises performing a PCR reaction with a library of genomic DNA molecules using a library primer that comprises a first Nextera adaptor sequence and a non-targeting primer that targets the vector backbone, wherein the non-targeting primer comprises a vector-specific sequence and a second Nextera adaptor sequence. In certain embodiments, the method comprises performing a PCR reaction with a library of genomic DNA molecules using a library primer that is a first Nextera adaptor sequence and a non-targeting primer that targets the vector backbone, wherein the targeting primer comprises a vector-specific sequence and a second Nextera adaptor sequence. In some embodiments, the first adaptor comprises a polynucleotide sequence of SEQ ID NO:9 or SEQ ID NO:11, the library primer comprises a polynucleotide sequence of SEQ ID NO: 2, the second adaptor sequence comprises a polynucleotide sequence of SEQ ID NO:10 or SEQ ID NO:12, and the targeting primer comprises a target specific sequence and a polynucleotide sequence of SEQ ID NO: 4. In other embodiments, the first adaptor comprises a polynucleotide sequence of SEQ ID NO:10 or SEQ ID NO:12, the library primer comprises a polynucleotide sequence of SEQ ID NO: 4, the second adaptor sequence comprises a polynucleotide sequence of SEQ ID NO:9 or SEQ ID NO:11, and the targeting primer comprises a target specific sequence and a polynucleotide sequence of SEQ ID NO: 2. In further embodiments, the amplification products are used as template for a subsequent round of PCR using the library primer that comprises the first Nextera adaptor sequence and a targeting primer that targets a specific gene or gene segment of interest, wherein the targeting primer comprises a target specific sequence and a second Nextera adaptor sequence.

In some embodiments, a library of double-stranded nucleic acid molecules is obtained from a subject, such as a human subject. In other embodiments, a plurality of nucleic acid molecules may be obtained from other subjects, including prokaryotic organisms, eukaryotic organisms, viruses, or viroids. Prokaryotic organisms include bacteria and archaea. Eukaryotic organisms include protozoa, algae, plants, slime molds, fungi (e.g., yeast), and animals. Animals include mammals, such as a primate, cow, dog, cat, rodent (e.g., mouse, rat, guinea pig), rabbit, or non-mammals, such as nematodes, bird, amphibian, reptile, or fish. A plurality of nucleic acid molecules may be from any sample from a subject, tissue or fluid, including blood, tumor biopsy, tissue biopsy, saliva, sputum, cerebral spinal fluid, vitreous fluid, vaginal secretion, semen sample, breast secretion, fecal sample, amniotic fluid, embryo biopsy, or urine. A sample may contain normal, abnormal (diseased, infected, damaged, affected) or both types of tissues or cells. A plurality of nucleic acid molecules may include nucleic acid molecules from more than one subject, such as nucleic acid molecules from a mother and fetus or nucleic acid molecules from host and infectious agent (e.g., virus, bacteria, fungi, protozoa, parasite that causes an infectious disease or infection in the host).

In some embodiments, the library of double-stranded nucleic acid molecules are obtained from a primary cell culture or culture adapted cell line including but not limited to genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines, stem cells, germ cells (e.g., sperm, oocytes), transformed cell lines and the like. For example, polynucleotide molecules may be obtained from primary cells, cell lines, freshly isolated cells or tissues, frozen cells or tissues, paraffin embedded cells or tissues, fixed cells or tissues, and/or laser dissected cells or tissues.

In some embodiments, a library of double-stranded nucleic acid molecules is obtained from an environmental sample. In certain embodiments, the environmental sample is a water sample or soil sample. In some embodiments, the environmental sample is derived from recreational water. “Recreational water” includes ocean water, pond water, lake water, creek water, river water, swimming pools, hot tubs, saunas, or the like. The methods described herein are also suited for use with other sample sources, including shellfish or other aquatic organisms, terrestrial organisms, groundwater, leachate, wastewater, sewer water, blackwater, graywater, bilge water, ballast water, feed water, process water, industrial water, irrigation water, rain water, runoff water, cooling water, non-potable water, potable water, drinking water, semi-pure water, spent ultra-pure water, or the like.

In some embodiments, a library of double-stranded nucleic acid molecules comprise a size ranging from about 15 to about 10,000 nucleotides, from about 15 to about 8,000 nucleotides, from about 15 to about 5,000 nucleotides, from about 15 to about 3,000 nucleotides, from about 50 to about 1000 nucleotides, from about 50 to about 500 nucleotides, or about 100 to about 300 nucleotides. In certain embodiments, a library of double-stranded nucleic acid molecules comprise an average size of about 750, about 500, about 400, about 300, about 250, about 200, about 150, about 100, or about 70 nucleotides.

In some embodiments, each double-stranded nucleic acid molecule or target nucleic acid molecule from a library of nucleic acid molecules contained within a vector is flanked by a 5′-barcode, a 3′-barcode, or by 3′- and 5′-barcodes. As used herein, the term “barcode” or “identifier tag” and variants thereof are used interchangeably and refer to a nucleic acid sequence that can be used to identify one or more particular nucleic acid molecules of interest. A unique barcode flanking a target nucleic acid molecule or a portion thereof can link each target nucleic acid molecule or portion thereof with each other and with the original complementary strand (e.g., before any amplification), so that each linked sequence serves as its own internal control. In other words, by uniquely tagging double-stranded nucleic acid molecules, sequence data obtained from one strand of tandem repeats of a single nucleic acid molecule can be compared within a strand and specifically linked to sequence data obtained from the complementary strand of that same double-stranded nucleic acid molecule.

In some embodiments, a barcode comprises a nucleic acid sequence of about 5 to about 50 nucleotides in length. In certain embodiments, all of the nucleotides of the barcode are not identical (i.e., comprise at least two different nucleotides) and optionally do not contain three contiguous nucleotides that are identical. In further embodiments, the barcodes used in the double-stranded nucleic acid molecule library comprise from about 5 nucleotides to about 40 nucleotides, about 5 nucleotides to about 30 nucleotides, about 6 nucleotides to about 30 nucleotides, about 6 nucleotides to about 20 nucleotides, about 6 nucleotides to about 10 nucleotides, about 6 nucleotides to about 8 nucleotides, about 7 nucleotides to about 9, or about 10 nucleotides, or about 6, about 7 or about 8 nucleotides. In particular embodiments, a barcode is comprised of about 6 to about 15 nucleotides or about 14, 13, 12, 11, 10, 9, 8, 7, 6, or 5 nucleotides.

The number of random nucleotides contained in each of the barcodes will govern the total number of possible barcodes available for use in a library. Shorter bar codes allow for a smaller number of unique barcodes, which may be useful when performing a deep sequence of one or a few nucleotide sequences, whereas longer bar codes may be desirable when examining a population of nucleic acid molecules, such as cDNAs or genomic fragments. For example, a barcode of 7 random nucleotides would have a formula of 5′-NNNNNNN-3′ (SEQ ID NO:5), wherein N may be any naturally occurring nucleotide. The four naturally occurring nucleotides are A, T, C, and G, so the total number of possible barcodes is 4⁷, or 16,384 possible random arrangements (i.e., 16,384 different or unique barcodes). For 6, 8, and 14 nucleotide bar codes, the number of random barcodes would be 4,096; 65,536; and 268,435,456, respectively. In certain embodiments of 6, 7, 8, or 14 random nucleotide barcodes, there may be fewer than the pool of 4,094, 16,384, 65,536, or 268,435,456 unique barcodes, respectively, available for use when excluding, for example, sequences in which all the nucleotides are identical (e.g., all A or all T or all C or all G) or when excluding sequences in which three contiguous nucleotides are identical or when excluding both of these types of molecules. In addition, the first about 5 nucleotides to about 20 nucleotides of the target nucleic acid molecule sequence may be used as a further identifier tag together with the sequence of an associated random barcode.

In any of the embodiments described herein, a barcode, besides a random sequence of nucleotides ranging in length from about 5 nucleotides to about 10 nucleotides, may further comprise a portion of about 2 nucleotides to about 25 nucleotides of a library nucleic acid molecule that is adjacent to or near the barcode on a library vector. In other words, in certain embodiments, a random barcode may further include a unique identifier comprised of a few junction nucleotides of the library nucleic acid molecule insert adjacent to or near the barcode. In certain embodiments, a random barcode may further comprise from about 5 nucleotides to about 20 nucleotides of a target nucleic acid molecule that is downstream or upstream of a barcode. In further embodiments, a random barcode further comprises from about 5 nucleotides to about 25 nucleotides of a “genomic shear point,” which refers to the portion of a sheared genomic library nucleic acid molecule inserted next to a barcode in a library vector.

In certain embodiments, a barcode may further comprise a restriction endonuclease site. In some embodiments, a barcode may further comprise a unique index sequence specific for a particular sample so that a library can be pooled with other libraries having different index sequences to facilitate multiplex sequencing (also referred to as multiplexing). In certain embodiments, an index sequence comprises a length ranging from about 4 nucleotides to about 25 nucleotides. In further embodiments, a barcode may further comprise an adaptor sequence comprising a length ranging from about 20 nucleotides to about 100 nucleotides, such as adaptor sequences that are useful for bridge amplification.

Exemplary compositions providing double-stranded nucleic acid molecule libraries comprising a plurality of nucleic acid molecules and a plurality of random barcodes, or a plurality of nucleic acid vectors comprising a plurality of random barcodes, or methods of use are described in U.S. Patent Application Publication No. US 2015/0024950, which libraries and barcodes are hereby incorporated by reference in their entirety.

In certain embodiments, each double-stranded nucleic acid molecule or target nucleic acid molecule from a library of nucleic acid molecules contained within a vector is flanked on one side by a strand index sequence (SIS). The non-complementary sequence on each strand of a strand index sequence may not be identical. The non-complementary region may comprise, for example, from 2 to about 30 nucleotides, 2 to about 25 nucleotides, 2 to about 20 nucleotides, 2 to about 15 nucleotides, 2 to about 10 nucleotides, 2 to about 5 nucleotides, about 3 to about 30 nucleotides, about 3 to about 25 nucleotides, about 3 to about 20 nucleotides, about 3 to about 10 nucleotides, about 3 to about 8 nucleotides, about 4 to about 30 nucleotides, about 4 to about 25 nucleotides, about 4 to about 20 nucleotides, about 4 to about 15 nucleotides, 4 to about 10 nucleotides, about 4 to about 8 nucleotides, or comprise 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. Each complementary region capable of forming a duplex structure may comprise, for example, from about 10 to about 50 nucleotides, about 10 to about 40 nucleotides, about 10 to about 35 nucleotides, about 10 to about 30 nucleotides, about 10 to about 25 nucleotides, about 10 to about 20 nucleotides, about 10 to about 15 nucleotides, or about 10, 11, 12, 13, 14, 15, 16, 17, 18, 19, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides, wherein the nucleotide sequence on the sense strand is complementary to the antisense strand.

In particular embodiments, a strand index sequence (SIS) comprises a non-complementary region containing from about 3 to about 10 nucleotides, and the complementary regions flanking the non-complementary region and each capable of forming a duplex structure contain from about 10 to about 20 nucleotides. In further embodiments, a strand index sequence (SIS) comprises a non-complementary region containing from about 5 to about 8 nucleotides, and the complementary regions flanking the non-complementary region and each capable of forming a duplex structure contain from about 10 to about 15 nucleotides.

In certain embodiments, the strand index sequence is positioned between a double-stranded nucleic acid molecule insert and the first adaptor sequence and barcode. Thus, in one example, if read in a 5′ to 3′ direction, the orientation can be 5′-first adaptor-barcode-strand index sequence-nucleic acid molecule insert-3′. Alternatively, the orientation can be 5′-nucleic acid molecule insert-strand index sequence-barcode-first adaptor-3′.

In certain embodiments, a strand index sequence may further comprise a restriction endonuclease site. A strand index sequence may include a restriction endonuclease site within one duplex region or both duplex regions. A restriction endonuclease site within a strand index sequence may be designed to match restriction endonuclease sites at the vector insertion site or other adjacent fragments (e.g., double-stranded nucleic acid molecule insert) in the final vector library construct.

In certain embodiments, a strand index sequence may comprise SEQ ID NO:7, SEQ ID NO:8, or both.

In certain embodiments, a targeting primer, a nested primer, or a non-targeting primer does not contain a full adaptor sequence. In other words, in some embodiments, the targeting primer comprises a truncated adaptor sequence. The adaptor sequence can be lengthened by a second PCR reaction that uses a primer that includes the full-length adaptor sequence. For example, the targeting primer can be a truncated targeting primer that comprises from 5 to 10 nucleotides of an adaptor sequence, which is used in a first PCR amplification reaction. The products of the first PCR amplification reaction can used in a second PCR reaction wherein the targeting primer comprises the full-length adaptor sequence (e.g., a 5′ adaptor “tail”). The product of the second PCR amplification reaction comprises a first adaptor sequence, a first barcode, a target nucleic acid molecule, and a second adaptor sequence. In certain embodiments, the PCR product can further comprise an index sequence.

Alternately, in certain embodiments, the targeting primer, non-targeting primer, or nested primer is a tailed targeting primer, tailed non-targeting primer, or tailed nested primer, respectively, wherein the tail comprises a second barcode or a portion thereof. The amplification product containing the targeting primer, non-targeting primer, or nested primer, and second barcode can be lengthened by a second PCR reaction using a third primer comprising the second barcode and a second adaptor sequence at its 5′ end. For example, the targeting primer can be a truncated targeting primer that comprises from 5 to 10 nucleotides of a second barcode, which is used in a first PCR amplification reaction. The first PCR amplification reaction can be followed by a second PCR reaction wherein the targeting primer comprises the full-length barcode and a second adaptor sequence. The product of the second PCR amplification reaction comprises a first adaptor sequence, a first barcode, a target nucleic acid molecule, a second barcode, and a second adaptor sequence. In certain embodiments, the PCR product can further comprise an index sequence.

In some embodiments, the library primer, the targeting primer, the non-targeting primer, the nested primer, or any combination thereof comprise a tag. In certain embodiments, the tag is an affinity tag. A tag, or affinity tag, comprises a detectable molecule (biological or chemical) that may allow for isolation or selection of its partner molecule to which the tag is attached (e.g., the products of target-specific primer-directed PCR) via interactions with a binding substrate for the tag. A tag allows for isolation or selection that is independent of the tagged partner molecule's structure or sequence. For example, an affinity tag can be used to purify the PCR product. Exemplary methods for purification include using a bead pull-down or affinity chromatography with the complimentary binding agent. Tag molecules may be attached using genetic methods or chemically coupled. For example, an amino modifier placed on the 5′ or 3′ end of the targeting primer can be used to tag the PCR product. Tag molecules are well known in the art and include, e.g., biotin, HIS tag, Flag® epitope, GST, chitin binding protein, maltose binding protein, HA-tag, Myc-tag, or the like. In certain embodiments, the tag is biotin. In certain embodiments, a primer is tagged at its 5′-end, 3′-end, an internal position, or a combination thereof. In further embodiments, following PCR amplification, biotin-tagged strands of tandem nucleic acid molecules comprising multiple copies of first target nucleic acid molecule or portion thereof are selected or isolated with streptavidin or avidin before a second amplification step. In further embodiments, methods described herein can be repeated with the library of double-stranded circular template molecules that have been separated from the biotin-tagged strands of tandem nucleic acid molecules.

In certain embodiments, products of a PCR reaction on a vector library of double-stranded template nucleic acid molecules are affinity purified before using the PCR products for a subsequent round of PCR. For example, a targeting primer is affinity tagged (e.g., biotinylated) and used with a library primer for an initial PCR reaction on a vector library of double-stranded template nucleic acid molecules. Biotinylated PCR products are purified (e.g., using streptavidin coated Dynabeads) from the rest of the PCR reaction components. The purified PCR products are used as template for a second PCR reaction using the library primer and a nested primer. In another example, a non-targeting primer is affinity tagged (e.g., biotinylated) and used with a library primer for an initial PCR reaction on a vector library of double-stranded template nucleic acid molecules. Biotinylated PCR products are purified (e.g., streptavidin coated Dynabeads) from the rest of the PCR reaction components. The purified PCR products are used as template for a second PCR reaction using the library primer and a targeting primer.

The methods described herein can be used to amplify and/or sequence more than one target nucleic acid molecule. In certain embodiments, the method comprises at least one or more targeting primers specific for at least a second target nucleic acid molecule. Accordingly, in some embodiments, the method comprises amplifying with the library primer and a plurality of targeting primers that are specific for a plurality of different target nucleic acid molecules. In certain embodiments, a plurality of different target nucleic acid molecules is from 2 to about 50, from 2 to about 100, from 2 to about 200, from 2 to about 300, from 2 to about 400, from 2 to about 500, or more different target nucleic acid molecules. The methods described allow for multiplex detection of mutations in multiple target nucleic acid molecules. In some embodiments, the methods described herein allow for whole genome or exome sequencing.

In some embodiments, a target nucleic acid molecule comprises a gene or nucleic acid sequence associated with or linked to a disease or disorder. The target nucleic acid molecule can comprise a fusion gene or a somatically mutated gene, which include a deletion, duplication, point mutation, insertion or any combination thereof. Genes associated with various diseases and disorders are known in the art and a number of these genes are provided in Tables A, B, and C of U.S. Pat. No. 8,795,965, which gene targets are herein incorporated by reference in their entirety.

In certain embodiments, a target nucleic acid molecule comprises all or a portion of a tumor suppressor gene, an oncogene, or any combination thereof. Exemplary target nucleic acid molecules include BCR-ABL, RAS, RAF, MYC, P53, ER (Estrogen Receptor), HER2, EGFR, mTOR, VEGF, ALK, pTEN, RB, DNMT3A, FLT3, NPM1, IDH1, IDH2, JAK, BRCA, APC, VHL, INK4, DPC4, MADR2, NF1, NF2, PTC, WT1, SRC, BCL, ERBB, MDM2, BRAF, ATM, AXIN2, BARD1, BRIP1, MRE11A, NBN, RAD50, BMPR1A, CDH1, CDH4, CDK4, CDKN2A, CHEK2, EPCAM, FANCC, GREM1, MLH1, MSH2, MSH6, MUTYH, NBN, PALB2, PMS2, POLD1, POLE, RAD51C, RAD51D, SCG5/GREM1, SMARCA4, SMAD4, STK11, XRCC2 or the like. In addition, a number of tumor suppressor genes and oncogenes that may be targets of the presently disclosed methods are provided in Futreal et al., Nature Reviews Cancer 4:177-83, 2004, which is incorporated by reference in its entirety. In addition, cancer related genes can be found in the “Catalogue of Somatic Mutations in Cancer” database maintained by the Sanger Institute (cancer.sanger.ac.uk/cancergenome/projects/census), which is herein incorporated by reference. For example, the methods described here may be used to monitor mutational spectrum of tumor suppressor genes or oncogenes in a sample from a subject.

In certain embodiments, identification of certain target nucleic acid molecule mutations would reveal a population of subjects for which one or more medications (such as imatinib, vemurafenib, tamoxifen, toremifene, traztuzumab, lapatinib, cetuximab, panitumumab, rapamycin, temsirolimus, everolimus, vandetanib, bevacizumab, crizotinib) known to provide a therapeutic or prophylactic effect could be chosen for treatment of that specifically identified population of subjects, or are not chosen when it is known the one or more medications fail to provide a therapeutic or prophylactic effect to the specifically identified population of subjects.

In certain embodiments, the target nucleic acid molecule comprises a coding sequence, non-coding sequence, or gene associated with a metabolic disorder, neurological disorder, immune disorder, developmental disorder, aging or genetic disorder.

In further embodiments, a target nucleic acid molecule comprises a coding sequence, non-coding sequence, or gene associated with a pathogenic organism (e.g., a virus, bacteria, or parasite), commensal organism, forensic testing, or any combination thereof.

Upon amplification of a target nucleic acid as described herein, the amplification product is sequenced. In some embodiments, sequencing is sequencing by synthesis, pyrosequencing, reversible dye-terminator sequencing, polony sequencing, semiconductor sequencing, or single molecule (e.g., nanopore) sequencing.

In any of the embodiments described herein, the sequencing can further comprise alignment of the sequences of each first target nucleic acid molecule or a portion thereof having matching barcodes, and wherein the alignment results in a consensus sequence with a measureable sequencing error rate equal to or a less than 10⁻⁶. For example, each copy of the first target nucleic acid molecule or portion thereof, presented on a strand (or multiple strands) produced by PCR amplification can be identified by its unique 5′ or 3′ barcode. Individual sequence reads containing the same barcode are grouped into read families, and consensus sequences are derived. In certain embodiments, a read family has at least three members before a consensus sequence is derived. These sequences may be aligned, and a mutation may be distinguished as a polymerase error artifact or a true mutation by a person of skill in the art. Since mutation introduced by PCR error will not be found the majority of PCR produces and the error rate is predictable, a true mutation in a target nucleic acid molecule is likely to be present in all of the copies present, which may be identified by their unique barcodes. In certain embodiments, a mutation in a target nucleic acid molecule is “called” (considered real and not an artifact) if it is observed in two or more read families.

In certain embodiments, the methods of this instant disclosure will be useful in detecting rare mutants against a large background signal, such as when monitoring circulating tumor cells; detecting circulating mutant DNA in blood, detecting fetal DNA in maternal blood, monitoring or detecting disease and rare mutations by direct sequencing, monitoring or detecting disease or drug response associated mutations. Additional embodiments may be used to quantify DNA damage, quantifying or detecting mutations in infectious agents (e.g., during HIV and other viral infections) that may be indicative of response to therapy or may be useful in monitoring disease progression or recurrence. In yet other embodiments, these compositions and methods may be useful in detecting damage to DNA from chemotherapy, or in detection and quantitation of specific methylation of DNA sequences.

In some embodiments, the methods disclosed herein further comprise methods of preparing a vector comprising a double stranded nucleic acid molecule library, which contains one or more target nucleic acid molecules. The methods can be used to prepare more than one vector to generate one or more libraries. For example, a collection of nucleic acid molecules representing the entire genome is called a genomic library. Methods for construction of nucleic acid molecule libraries are well known in the art (see, e.g., Current Protocols in Molecular Biology, Ausubel et al., Eds., John Wiley & Sons, New York, 2003; Current Protocols in Molecular Biology, Ausubel et al., Eds., Greene Publishing and Wiley-Interscience, New York, 1995; Sambrook et al., Molecular Cloning: A Laboratory Manual, 2nd Ed., Cold Spring Harbor Laboratory Vols. 1-3, 1989; Methods in Enzymology, Vol. 152, Guide to Molecular Cloning Techniques, Berger and Kimmel, Eds., San Diego: Academic Press, Inc., 1987).

Accordingly, in certain embodiments, the method of any of the embodiments described herein further comprises the step of generating a library of double-stranded circular template molecules comprising, (a) isolating DNA from a sample, (b) fragmenting the DNA, and (c) inserting the fragmented DNA and optionally a strand index sequence into a vector, wherein the vector comprises the barcode, the first adaptor region, and the first priming site.

Once isolated from a sample, a plurality of nucleic acid molecules may undergo further processing prior to cloning into vectors. Such processing includes fragmentation by processes such as mechanical shearing, cleavage with restriction endonucleases, enzymatic fragmentation, or a combination thereof, to generate shorter nucleic acid molecule fragments. In some embodiments, isolated nucleic acid molecules do not require processing because they are naturally sheared (e.g., plasma DNA). Nucleic acid fragments having overhanging ends may be repaired (i.e., blunted or “polished”) using T4 DNA polymerase and E. coli DNA polymerase I Klenow fragment. Ribonucleic acid molecules may undergo reverse transcription and cDNA synthesis to produce a plurality of double-stranded nucleic acid molecules for insertion into the vectors. A synthesis step may be performed on single stranded nucleic acid molecules to produce a plurality of double-stranded nucleic acid molecules for insertion into the vectors. In some embodiments, 3′ A-overhangs are added to the plurality of double-stranded nucleic acid molecules. In some embodiments, 3′ A-overhangs are added to one end (5′ or 3′) or both ends of the plurality of double-stranded nucleic acid molecules. The 3′ A-overhangs can be added using, for example, Taq polymerase.

In certain embodiments, double-stranded nucleic acid molecules are cloned into vectors, with a 5′ first adaptor sequence and a unique barcode or a 3′ first adaptor sequence and a unique barcode flanking the cloning site on one side. The double-stranded nucleic acid molecules, which are the nucleic acid molecules of interest for amplification and sequencing, may range in size from a few nucleotides (e.g., 15) to many thousands (e.g., 10,000). Preferably, the double-stranded nucleic acid molecules in the library range in size from about 100 nucleotides to about 3,000 nucleotides, from about 100 nucleotides to about 600 nucleotides, or from about 100 nucleotides to about 300 nucleotides. In some embodiments, the double-stranded nucleic acid molecules are average size of about 100 nucleotides, about 150 nucleotides, about 200 nucleotides, about 250 nucleotides, or about 300 nucleotides.

In some embodiments, a plurality of double-stranded nucleic acid molecules are inserted (e.g., cloned) into a plurality of vectors to form a library of double-stranded circular template molecules. The plurality of double stranded nucleic acid molecules can be inserted into the vector using, for example, T4 DNA ligase. In some embodiments, a vector comprises at least one XcmI restriction site (e.g., XcmI restriction site and another restriction site) for insertion of double stranded nucleic acid molecules (e.g., genomic DNA). In some embodiments, a vector comprises two XcmI restriction sites for insertion of double stranded nucleic acid molecules. In other embodiments, a vector comprises at least one (one, two, or more) restriction site for enzymes other than XcmI. For example, if a vector comprises two XcmI restriction sites, digest of the vector with XcmI will yield a linear vector with a T-overhang on each strand. A plurality of double-stranded nucleic acid molecules is fragmented, blunted, and “A-tailed” to add a non-templated adenine to the 3′ end of each strand using Taq polymerase. The T-tailed vector and A-tailed inserts are ligated together using DNA ligase to produce a vector library of double-stranded circular template molecules. In another example, a vector comprises an XcmI restriction site and a restriction site for a second endonuclease enzyme, and digest of the vector with XcmI and the second endonuclease will yield a linear vector with a T-overhang on one end from the XcmI site and a non-complementary overhang on the other end from the second endonuclease. A plurality of double-stranded nucleic acid molecules having a A-overhang on one end and a second overhang to match the T-overhang and the non-complementary overhang from the second endonuclease in the vector, respectively, are ligated together using DNA ligase to produce a vector library of double-stranded circular template molecules. In yet another example, a strand index sequence is incorporated into a vector comprising an XcmI restriction site and a restriction site for a second endonuclease enzyme. Digest of the vector with XcmI and the second endonuclease will yield a linear vector with a T-overhang on one end from the XcmI site and a non-complementary overhang on the other end from the second endonuclease. A plurality of double-stranded nucleic acid molecules is fragmented, blunted, and “A-tailed” to add a non-templated adenine to the 3′ end of each strand using Taq polymerase. A plurality of strand index sequences is prepared having one 3′ T-overhang and an overhang at the other end that complements the vector overhang from the second endonuclease. The vector, plurality of double-stranded nucleic acid molecules, and plurality of strand index sequences are ligated together using DNA ligase to produce a vector library of double-stranded circular template molecules having a strand index sequence. Thus, the orientation of resulting vector library can be 5′-restriction enzyme 2-strand index sequence-nucleic acid molecule insert-XcmI-3′, or 5′-XcmI-nucleic acid molecule insert-strand index sequence-restriction enzyme 2-3′.

Double stranded nucleic acid molecule libraries comprising a plurality of nucleic acid molecules and a plurality of random barcodes, nucleic acid vector libraries comprising a plurality of random barcodes, and methods of use are described in U.S. Patent Application Publication No. US 2015/0024950, which libraries and barcodes are hereby incorporated by reference in their entirety.

An advantage of the methods described herein is that upon insertion of a double-stranded nucleic acid molecule, the vector is ready for an amplification step (e.g., PCR) without transformation and selection in a host organism (e.g., bacteria, yeast, or cell line). Therefore, completing the library preparation and enrichment in a single step decreases the time to sequencing from about 3-4 days for existing technology to a few hours with the methods provided herein. In some embodiments, the amplification step is performed directly after the vector or library preparation step. However, if desired, the vector can be cloned into a host (e.g., E. coli) prior to amplification.

In some embodiments, prior to insertion of a double-stranded nucleic acid molecule, the vector may comprise an insert in the cloning site. Such an insert may comprise a nucleic acid molecule that encodes a marker, such as a fluorescent protein (GFP, RGP, YFP, CFP, etc.), lacZ, lacZα, or the like. Such an insert may comprise a nucleic acid molecule that encodes a selective marker, such as toxic agents like Control of Cell Death B protein (ccdB), Bacillus subtilis levansucrase (sacB), or the like. The insert can be used to differentiate empty vectors from vectors that have had a double-stranded nucleic acid or target nucleic acid successfully cloned into the vector.

It is contemplated that a kit would be useful for carrying out the methods described herein. Accordingly, in some embodiments, is a kit comprising a vector comprising a first priming site, a first adaptor sequence, a barcode, and a restriction enzyme cleavage site; a library primer, and at least one targeting primer or a non-targeting primer, wherein the targeting primer or non-targeting primer optionally comprises a second adaptor sequence at its 5′ end. In certain embodiments, at least one targeting primer targets a sequence within a gene associated with cancer. In some embodiments, the targeting primer targets a gene selected from BCR-ABL, RAS, RAF, MYC, P53, ER (Estrogen Receptor), HER2, EGFR, mTOR, VEGF, ALK, pTEN, RB, DNMT3A, FLT3, NPM1, IDH1, IDH2, JAK, BRCA, PTEN, APC, VHL, INK4, DPC4, MADR2, NF1, NF2, PTC, WT1, SRC, BCL, ERBB, MDM2, BRAF, ATM, AXIN2, BARD1, BRIP1, MRE11A, NBN, RAD50, BMPR1A, CDH1, CDH4, CDK4, CDKN2A, CHEK2, EPCAM, FANCC, GREM1, MLH1, MSH2, MSH6, MUTYH, NBN, PALB2, PMS2, POLD1, POLE, RAD51C, RAD51D, SCG5/GREM1, SMARCA4, SMAD4, STK11, XRCC2 or a combination thereof. In some embodiments of the kit, the library primer has the sequence of SEQ ID NO: 2 or SEQ ID NO:4. In some embodiments of the kit, the targeting primer has the sequence of SEQ ID NO: 3.

### EXAMPLES

### Example 1

### Vector Preparation and Sequencing

The following experiments were performed to test the efficiency of the method of vector preparation and subsequence sequencing reaction.

### Sample Preparation for Cloning

Human genomic DNA was extracted and subsequently fragmented to an average size of 150 bp per fragment. The fragments were gel-purified to select fragments between the size of 100 to 300 bp. The fragments were then blunted using a blunting kit from NEB. 3′ A-overhangs were added using Taq polymerase and dATP. The fragments were then ready for ligation.

### Preparation of Vector

The vector was prepared for ligation by digestion with the restriction enzyme XcmI, which results in 3′ T-overhangs on either end of the linearized vector. The vector was gel purified after digestion to remove any undigested vectors. The vector and DNA fragments were mixed and ligated together with DNA ligase. The efficiency of this ligation was quite high due to the T and A overhangs on the vectors and fragments, respectively. The ligation produced a short-read plasmid library of the original DNA sample.

### Target Enrichment

In this example the target nucleic acid was p53 exon 4. In order to simultaneously enrich for a target region, the plasmid library was used as a template for a PCR reaction using one Nextera library primer (SEQ ID NO: 2) and a targeting primer. The targeting primer included a second Nextera adaptor sequence 5′ to a sequence matching the target locus, p53 exon 4 (SEQ ID NO: 3). Only plasmids containing p53 exon 4 DNA that matches the targeting primer were amplified exponentially resulting in specific enrichment of the target. The inclusion of the Nextera adaptor sequence on the targeting primer allowed the enrichment PCR to produce sequencing-ready substrates without any further steps required. The PCR products were quantified using droplet digital PCR with the Nextera library primers, denatured and diluted according to the Illumina standard protocol. The samples were run on an Illumina-based next generation sequencing system.

### Example 2

### Enrichment PCR

The following experiments were performed to test the specificity of the PCR enrichment protocol.

A sequencing library from HeLa S3 cell DNA was prepared using the methods described in Example 1. A sequencing library was independently prepared for a p53 exon 4 sequence. Three samples were made: 12.5 ng HeLa library alone, 12.5 ng HeLa library spiked with 0.125 pg p53 library (0.001% of the total input), and 0.125 pg p53 library alone.

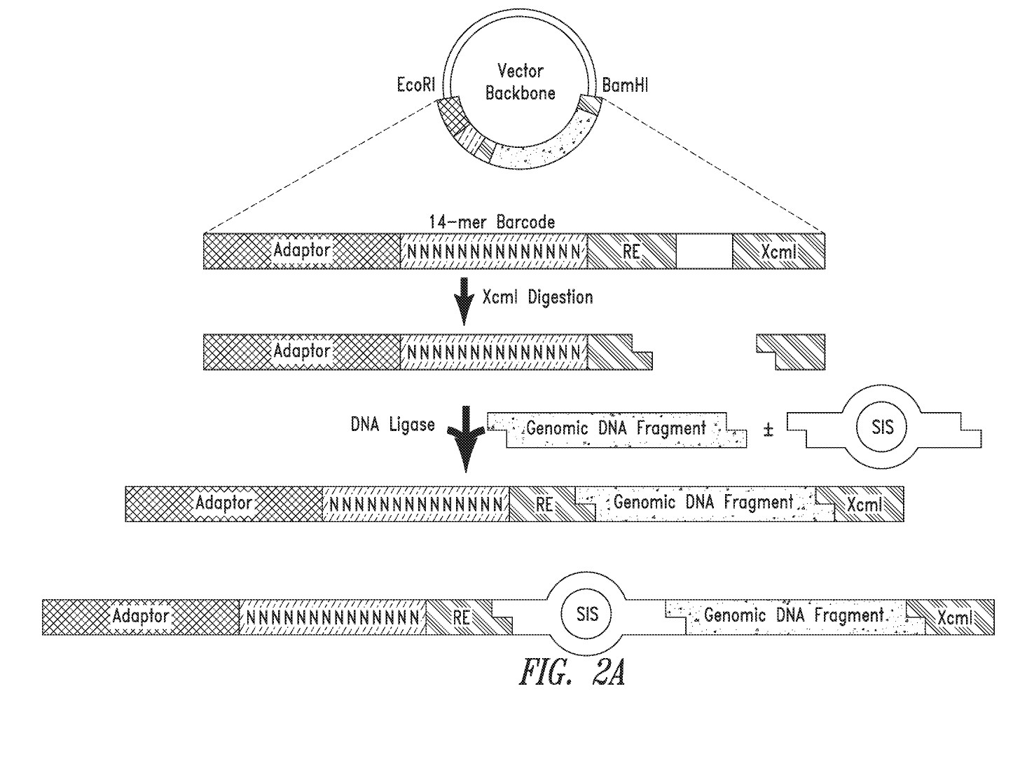

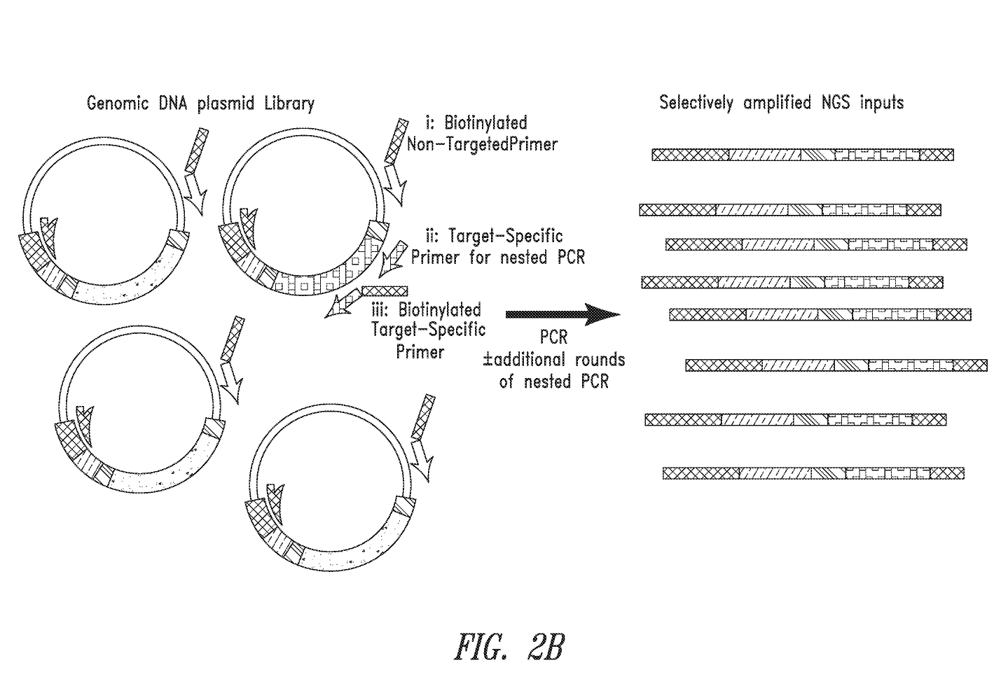

These samples were PCR amplified with the Library Primer and Targeting Primer. As shown FIG. 2 for a HeLa library alone very little PCR product was amplified. The mixture of the HeLa library and the small amount of p53 exon 4 library amplification resulted in strong band at the predicted size for p53 exon 4 amplicons and matched the positive control amplicon of the p53 exon 4 library alone.

In addition, the PCR products were digested with NcoI, which is present in the p53 exon 4 sequence. The PCR products were cleaved into the expected sizes for p53 exon 4 (FIG. 2). This demonstrates that specific targets, such as p53 exon 4, can be selectively amplified and enriched, for sequencing. From this result we calculate approximately 90% or more of the products are the desired p53 exon 4 targets, which corresponds to about a 100,000-fold enrichment for our target.

These results demonstrate that PCR enrichment of the target sequence is efficient and specific using the library primer and a p53 exon 4 specific target primer.

### Example 3

### Enrichment and Sequencing Data from HCT116 Cell Library

A sequencing library from HCT116 cell DNA was prepared using the methods described in Example 1. A sequencing library for p53 exon 5 and exon 6 from HCT116 cell DNA was also prepared. HCT116 genomic DNA was PCR amplified with primers flanking exons 5 and 6 of p53, SEQ ID NO:18 and SEQ ID NO:19. The p53 exon 5-exon6 amplicons, having 3′ A-overhangs added during the PCR reaction with Taq polymerase, were ligated into vector digested with the restriction enzyme XcmI as described in Example 1.

Two samples were prepared: (1) HCT116 library alone, and (2) HCT116 library spiked with the p53 library (one copy per approximately 5 million HCT116 copies). These samples were used as a template for a PCR reaction using one Nextera Library Primer (SEQ ID NO:2) and a biotinylated targeting primer (SEQ ID NO:19). The biotinylated targeting primer did not include a second Nextera adaptor sequence. Only templates containing p53 DNA that matched the targeting primer were amplified. After affinity purification of the amplicons using the biotin tag, the purified amplicons were used as a template for a second PCR reaction using the Nextera Library Primer (SEQ ID NO:2) and a nested targeting primer (SEQ ID NO:20) containing p53 exon 5 specific sequence and the Nextera read primer at its 5′ end. The amplicons from the nested PCR reaction were used as a template for a third PCR reaction using the Nextera Library Primer (SEQ ID NO:2) and two different Nextera index primers (SEQ ID NO:21 and SEQ ID NO:22), one for each sample, to append unique read indexes and allow for multiplex sequencing of the two samples. The Nextera index primers have from a 5′ to 3′ direction: (i) a flow adaptor sequence, (ii) index sequence, and (iii) a fragment of the 5′-end of read primer sequence from SEQ ID NO:20.

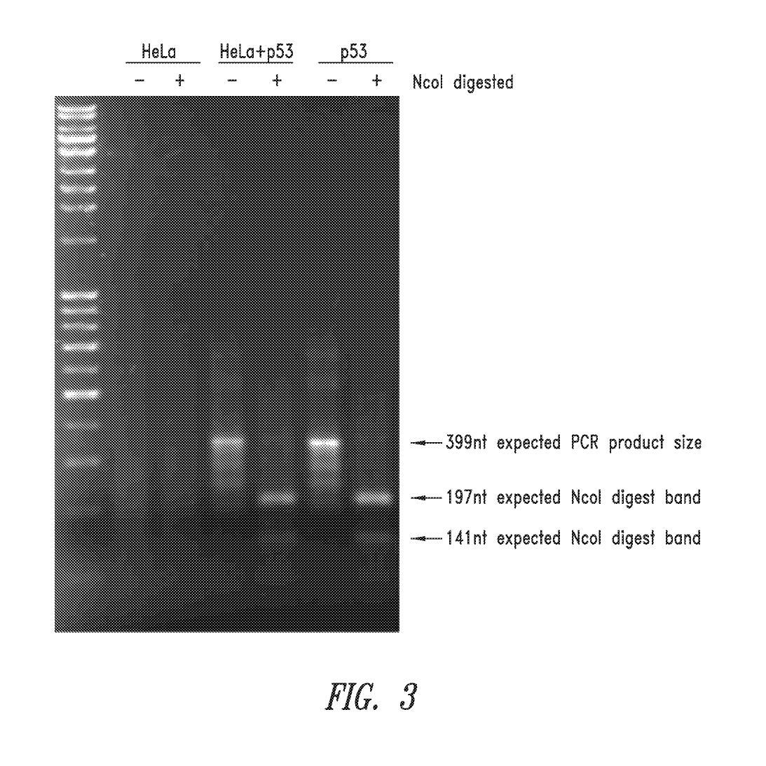

The enrichment of the resulting libraries was quantified by droplet digital PCR (ddPCR) and by sequencing and alignment to the human genome (see, FIG. 3). Both analyses showed a 1- or 2-million-fold enrichment for the HCT116 or HCT116+p53-spike, respectively, sufficient to enrich endogenous p53 copies to represent approximately 30% of all reads sequenced. These values were calculated based on an initial abundance of one p53-containing DNA fragment for every 6 million 500-base pair genomic DNA fragments per genome (i.e., 3 billion base genome divided by the sheared length of 500 bases).

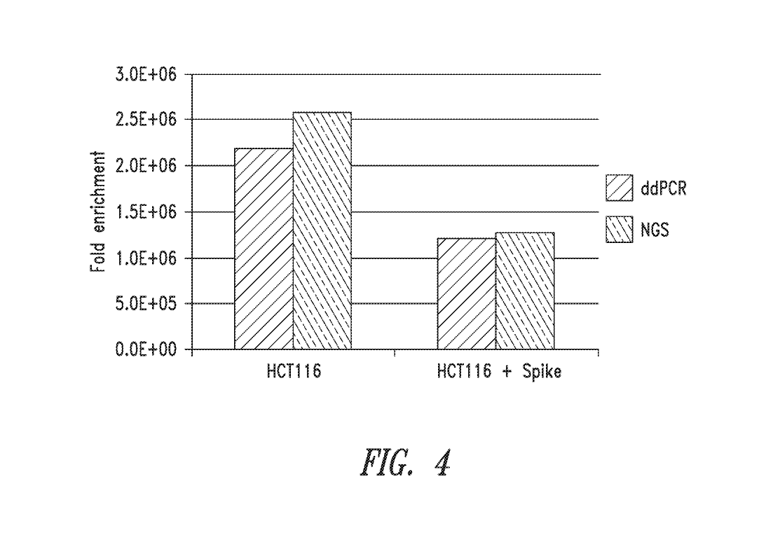

Double-stranded bar codes present within the sequencing reads were used to create a consensus sequence from each read family. A mutation was called if it was observed in two or more read families. In the sequencing data for the two libraries, two mutations were observed: (1) a SNP present in both datasets at a frequency of 1, and (2) a subclonal substitution present at a frequency of 0.001, which occurred only in the HCT116+p53-spike sample (see, FIG. 4). The subclonal mutation was determined to have been present in the PCR spike-in and not to be endogenous. This substitution was likely a mutation created during the PCR preparation of the p53 spike-in sequence. Excluding the p53 spike-in substitution artifact, no sequencing errors were present in the enriched p53 genomic region queried, as expected for the wild type HCT116 cells.

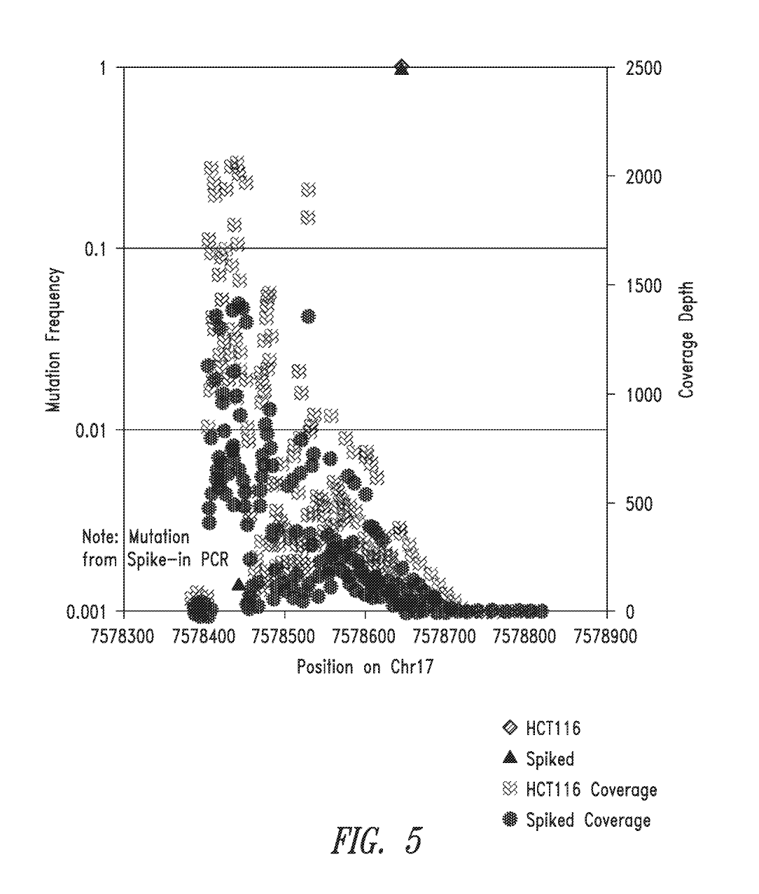

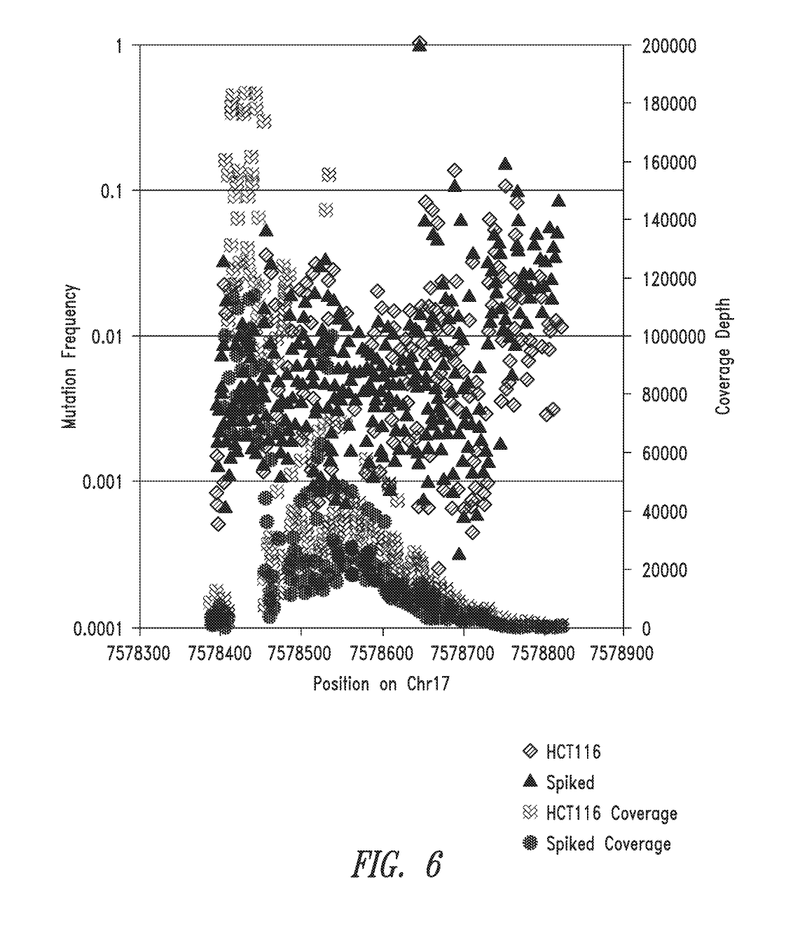

For comparison, the sequencing data for the two libraries was also processed without the benefit of error correction using the double-stranded bar codes (see, FIG. 5). The error rate without correction is substantially higher at nearly 1 error in every 100 base calls, resulting in errors called at nearly every position of the enriched p53 target locus.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to U.S. Provisional Patent Application No. 62/140,930 filed on Mar. 31, 2015, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for detecting mutations in a nucleic acid molecule, comprising amplifying a template target nucleic acid molecule from a vector library of double-stranded template nucleic acid molecules with a plurality of primers,
wherein each vector comprises a first adaptor and a barcode adjacent to the double-stranded template nucleic acid molecule insert and at least one vector of the library comprises a template target nucleic acid molecule; and
wherein the plurality of primers comprises: (i) a library primer comprising a sequence capable of hybridizing to a first priming site within or near the first adaptor and a targeting primer comprising a sequence capable of hybridizing to a target region within the target nucleic acid molecule; or (ii) the library primer and a non-targeting primer comprising a sequence capable of hybridizing to a second priming site adjacent to the double-stranded template nucleic acid molecule insert and on the opposite end from the first adaptor and barcode;
thereby producing an amplified target nucleic acid molecule comprising a first adaptor region and a barcode adjacent to a target nucleic acid molecule, which amplified target nucleic acid molecule can be sequenced using the plurality of primers to identify or detect mutations present in the template target nucleic acid molecule.

2. The method of claim 1, wherein the first adaptor comprises all or a portion of the first priming site.

3. The method of claim 1 or 2, wherein the library primer is complementary to the first adaptor.

4. The method of any one of claims 1-3, wherein the second priming site is within the vector backbone.

5. The method of any one of claims 1-4, wherein the method comprises amplifying with the library primer and the non-targeting primer.

6. The method of any one of claims 1-3, wherein the method comprises amplifying with the library primer and the targeting primer.

7. The method of any one of claims 1-6, wherein the targeting primer or non-targeting primer further comprises a second adaptor or a portion thereof at its 5′-end.

8. The method of claim 7, wherein the amplified target nucleic acid molecule produced comprises a target nucleic acid molecule flanked by the first adaptor region and the barcode on one end and the second adaptor region on the other end.

9. The method of any one of the preceding claims, wherein the library of double-stranded template nucleic acid molecules comprises genomic DNA or mitochondrial DNA.

10. The method of any one of claim 1-8, wherein the library of double-stranded template nucleic acid molecules comprises cDNA.

11. The method of any one of the preceding claims, wherein the library of double-stranded template nucleic acid molecules are from a subject or an environmental sample.

12. The method of claim 11, wherein the subject is a human.

13. The method of claim 11 or 12, wherein the double-stranded template nucleic acid molecules are obtained from a tumor sample, blood sample, biopsy sample, amniotic fluid sample, semen sample, urine sample, or fecal sample.

14. The method of claim 11, wherein the environmental sample is a water sample or soil sample.

15. The method of any one of the preceding claims, wherein each of the double-stranded template nucleic acid molecules in the library range in size from about 15 to about 3,000 nucleotides.

16. The method of any one of the preceding claims, wherein each of the double-stranded nucleic acid molecules in the library range in size from about 100 to about 300 nucleotides.

17. The method of any one of the preceding claims, wherein the barcode ranges in size from about 5 nucleotides to about 10 nucleotides.

18. The method of any one of the preceding claims, wherein the barcode further comprises a genomic shear point.

19. The method of any one of the preceding claims, wherein the method comprises amplifying with the library primer and a plurality of targeting primers that are specific for a plurality of different target nucleic acid molecules.

20. The method of claim 19, wherein the plurality of different target nucleic acid molecules is 2 to about 100 different target nucleic acid molecules.

21. The method of any one of claims 1-13 and 15-20, wherein the target nucleic acid molecule comprises a tumor suppressor gene, an oncogene, or any combination thereof.

22. The method of claim 21, wherein the target nucleic acid molecule comprises a sequence corresponding to a section of a p53 gene, Her2/neu gene, BRCA gene, Ras, RB, or a combination thereof.

23. The method of any one of claims 1-13 and 15-22, wherein the target nucleic acid molecule comprises a gene associated with a metabolic disorder, neurological disorder, immune disorder, developmental disorder, genetic disorder, pathogenic organism, commensal organism, forensic testing, or a combination thereof.

24. The method of any one of the preceding claims, wherein the sequencing is sequencing by synthesis, pyrosequencing, reversible dye-terminator sequencing, polony sequencing, semiconductor sequencing or single molecule sequencing.

25. The method of any one of claims 1-24, wherein the first adaptor is located 5′ of the barcode and the barcode is located 5′ of the target nucleic acid molecule.

26. The method of any once of claims 1-24, wherein the first adaptor is located 3′ of the barcode and the barcode is located 3′ of the target nucleic acid molecule.

27. The method of any one of the preceding claims, wherein the first adaptor is SEQ ID NO:9 and the second adaptor is SEQ ID NO:10, the first adaptor is SEQ ID NO:11 and the second adaptor is SEQ ID NO:12, the first adaptor is SEQ ID NO:10 and the second adaptor is SEQ ID NO:9, or the first adaptor is SEQ ID NO:12 and the second adaptor is SEQ ID NO:11.

28. The method of claim 27, wherein:
the first adaptor is SEQ ID NO:9 or SEQ ID NO:11, the second adaptor is SEQ ID NO:10 or 12, and the library primer is SEQ ID NO:2; or
the first adaptor is SEQ ID NO:10 or SEQ ID NO:12, the second adaptor is SEQ ID NO:9 or 11, and the library primer is SEQ ID NO:4.

29. The method of any one of the preceding claims, wherein the vector further comprises an index sequence.

30. The method of any one of the preceding claims, wherein the targeting primer or non-targeting primer comprises a truncated adaptor sequence at its 5′ end.

31. The method of any one of the preceding claims, wherein the amplified target nucleic acid molecule is used as a template for a subsequent amplification step with the library primer and a nested targeting primer comprising a sequence capable of hybridizing to a target region within the target nucleic acid molecule that (i) does not overlap with the target region of the targeting primer, or (ii) partially overlaps with the target region of the targeting primer at the 5′-end of the nested targeting primer.

32. The method of claim 31, wherein the nested targeting primer further comprises a second adaptor or a portion thereof at its 5′-end.

33. The method of any one of the preceding claims, wherein the library primer, the targeting primer, the non-targeting primer, the nested primer, or any combination thereof comprises a tag.

34. The method of claim 33, wherein the tag is an affinity tag.

35. The method of claim 34, wherein the affinity tag is biotin, a FLAG-tag, a HIS-tag, a HA-tag, or a Myc-tag.

36. The method of any one of the preceding claims, wherein the vector further comprises a strand index sequence comprising a non-complementary sequence flanked by duplex regions, wherein the strand index sequence is adjacent to the double-stranded template nucleic acid molecule insert.

37. The method of claim 36, wherein the non-complementary region has about 3 nucleotides to about 10 nucleotides, or about 5 nucleotides to about 8 nucleotides.

38. The method of claim 36 or 37, wherein each of the complementary regions capable of forming duplex structures has from about 10 nucleotides to about 25 nucleotides, about 10 nucleotides to about 20 nucleotides, or about 10 nucleotides to about 15 nucleotides.

39. The method of any one of claims 36-38, wherein the strand index sequence is located 5′ to the double-stranded template nucleic acid molecule insert.

40. The method of any one of claim 36-38, wherein the strand index sequence is located 3′ to the double-stranded template nucleic acid molecule insert.

41. The method of any one of claims 36-40, wherein the strand index sequence is flanked by the first adaptor and barcode on one end and the double-stranded template nucleic acid molecule insert on the other end.

42. The method of any of the preceding claims, wherein the sequencing further comprises alignment of the sequences of each amplified target nucleic acid molecule or a portion thereof having matching barcodes, and wherein the alignment results in a consensus sequence with a measureable sequencing error rate equal to or a less than 10⁻⁶.

43. The method of any one of claims 36-42, wherein the strand of the double-stranded template nucleic acid molecule from which the sequence of the amplified target nucleic acid molecule originated from may be identified based on the non-complementary sequence of the strand index sequence.

44. A kit, comprising: a vector, a library primer, and at least one targeting primer or non-targeting primer, wherein the targeting primer or non-targeting primer comprises a second adaptor at its 5′-end.

45. The kit of claim 44, wherein the vector comprises a first priming site for the library primer, a first adaptor sequence, a bar code, and at least one restriction enzyme cleavage site.

46. The kit of claim 44 or 45, wherein at least one targeting primer targets a sequence within a gene associated with cancer.

47. The kit of any one claims 44-46, wherein the targeting primer targets a gene selected from p53, Her2/neu, BRCA, Ras, RB, or a combination thereof.
